(19) United States
(12) Patent Application Publication
(10) Pub. No.: US 2014/0038197 A1
(43) Pub. Date: Feb. 6, 2014

(54) SYSTEM FOR AND METHOD OF DETERMINING CANCER PROGNOSIS AND PREDICTING RESPONSE TO THERAPY

(71) Applicants: Scott A. Waldman, Ardmore, PA (US); Theresa Hyslop, Glenside, PA (US)

(72) Inventors: Scott A. Waldman, Ardmore, PA (US); Theresa Hyslop, Glenside, PA (US)

(73) Assignee: Thomas Jefferson University, Philadelphia, PA (US)

(21) Appl. No.: 13/978,680

(22) PCT Filed: Jan. 9, 2012
(86) PCT No.: PCT/US12/20688

§ 371 Date: Oct. 22, 2013

Related U.S. Application Data
(60) Provisional application No. 61/430,887, filed on Jan. 7, 2011.

(51) Int. Cl. C12Q 1/68; G06F 19/00

(52) U.S. Cl. CPC C12Q 1/6886; G06F 19/3431
USPC 435/6.12; 705/3

(57) ABSTRACT

A database for predicting clinical outcomes based upon quantitative tumor burden in lymph node samples from an individual is provided. The database comprises data sets from a plurality of individuals. The data sets include clinical outcome data and data regarding number of lymph nodes evaluated, maximum number of biomarker detected in any single node, median normalized expression levels detected across all evaluated lymph nodes and the maximum normalized expression levels detected in any evaluated lymph nodes and the database also includes stratified risk categories based upon recursive partitioning of data. A system for predicting clinical outcomes based upon quantitative tumor burden in lymph node samples from an individual is provided which includes the database linked to a data processor, an input interface and an output interface. Method of preparing a database and method for predicting clinical outcome for a test patient based upon quantitative tumor burden in lymph node samples from an individual using a system that includes the database linked to a data processor, an input interface and an output interface. The method comprises measuring quantitative tumor burden in a plurality of lymph node samples from an individual, inputting the results into the system and processing with data in the database. The results of the processing of the data is the assignment of data test patient to a stratified risk category. Output is produced that displays test patient's identity and assigned stratified risk category.

This application claims priority to U.S. Provisional Application 61/430,887 filed Jan. 7, 2011, which is incorporated herein by reference.

Work associated with this invention was supported in part by grants from NIH (CA75123, CA95026 and CA112147). The United States government may have certain rights.

### FIELD OF THE INVENTION

The present invention related to and kits, compositions and systems and methods using the same to more accurately and precisely determine and establish a prognosis of an individual diagnosed with cancer, and to more accurately and precisely predict responses to therapy.

### BACKGROUND OF THE INVENTION

Metastasis of tumor cells to regional lymph nodes is among the most important prognostic factors in patients with many types of cancer. Recurrence rates vary widely between patients with lymph nodes deemed free of tumor cells by histopathology (pN0) and patients with histopathologically evident lymph node metastases. For example, in patients diagnosed with colorectal cancer, recurrence rates increase from approximately 25% in patients whose lymph nodes are determined to be free of tumor cells by histopathology (pN0) to approximately 50% in patients who are identified as having ≧4 lymph nodes harboring metastases as detected by histopathology.

Adjuvant chemotherapy improves disease-free and overall survival in patients with histopathologically evident lymph node metastases, but its role in pN0 patients remains unclear. In many cases, the standard treatment for pN0 patients is a wait and see. In patients diagnosed with colorectal cancer, such a wait and see approach may be followed among colorectal cancer pN0 patients despite knowing that 25% will have recurrent diseases.

Given the established relationship between lymph node metastasis and prognosis in many cancers, recurrence in a substantial minority of pN0 patients suggests the presence of occult lymph node metastases in regional lymph nodes that escape histopathological detection. The presence of occult lymph node metastases in regional lymph nodes from patients identified as being pN0 may be identified by the detection in lymphnodes of the presence of or elevated amounts of cancer associated molecular biomarkers such as proteins or mRNA encoding proteins which are expressed by cancer cells but either not normally found in lymph nodes or found at baseline or background levels. Patients identified as being pN0 which contain molecular biomarkers whose presence or elevated quantities in lymph nodes are referred to herein as pN0(mol+). Conversely, pN0 patients whose lymph nodes are free of molecular biomarkers or who contain molecular biomarkers at quantities consistent with normal lymph nodes are referred to herein as pN0(mol−). Patients identified as pN0(mol+) may be at elevated risk for developing recurrent disease while pN0(mol−) patients may be at lowest risk for developing recurrent disease.

The discovery of molecular techniques and systems for detecting occult lymph node metastases provides an additional diagnostic and predictive tool, particularly among those individuals deemed free of occult lymph node metastases (pN0) by histopathology examination. It is known that among such pN0 population, a proportion will experience disease recurrence and increased mortality levels.

Various technologies are known and may be used to molecularly analyze lymph node samples including but not limited to protein detection technologies such as PCR including, RT-PCR, quantitative PCR (qPCR), quantitative RT-PCR (qRT-PCR), immunohistochemistry using detectable binding agents, immunoassays such as ELISA or Western blots, nucleic acid detection technologies such as in situ hybridization using detectable probes (such as FISH), dot blots assays and Northern blots. Examples of these and other techniques are disclosed in U.S. Pat. No. 5,601,990, which is incorporated by reference, for example.

Methods using quantitative RT-PCR (qRT-PCR) to accurately measure the detection and quantitation of biomarker such as mRNA can be improved for detection of biomarker mRNA above background “noise” using an algorithm which standardizes the qRT-PCR data from different lymphnode samples to accommodate for variations of the qRT-PCR reactions among the different lymph node samples tested (see U.S. application Ser. No. 12/997,545 filed Apr. 22, 2011 which is the U.S. Nation Stage application of PCT Application PCT/US09/043,857 filed May 13, 2009 and published Nov. 19, 2009 as WO/2009/140436, which claims priority to U.S. Provisional Ser. No. 61/052,915 filed May 13, 2008, each of which is incorporated herein by reference).

The ability to differentiate pN0 patients as pN0(mol+) or pN0(mol−) provides additional insight in the likelihood of disease recurrence and thus provides additional information to determine if proceeding with adjunctive chemotherapy or following a wait and see approach is more appropriate. Patients deemed pN0 face a statistically risk of recurrence. Patients deemed pN0(mol+) or pN0(mol−) can be provided with a more accurate estimation of their statistical risk of recurrence. Patients deemed pN0(mol+) are more likely to recur than patients deemed pN0(mol−). Patients deemed pN0(mol+), however, may not suffer recurrence despite being pN0(mol+). Similarly, patients deemed pN0(mol−) may suffer recurrence despite being pN0(mol−). While not definitive, the ability to determine if a pN0 patient is pN0(mol+) or pN0(mol−) allows for decision making based upon improved statistics. Thus for example, a pN0 colorectal cancer patient statistically may a face 25% chance of recurrence but by determining if they are pN0(mol+) or pN0(mol−), the treating physician and patient will discover if that patient is actually at higher risk than 25% or a lower risk. Determining that the patient is at a risk higher than 25% may justify more aggressive treatment while determining that the patient is at a risk lower than 25% may alleviate some fear and stress in the patient as the wait and see whether they experience disease recurrence.

Determining pN0 patients as being pN0(mol+) or pN0(mol−) provides valuable predictive information regarding risk of recurrence. The likelihood of recurrence among pN0(mol+) is greater that that of the pN0 patient population as a whole. The identification of a group of patients as being pN0(mol+) allows for better allocation of risk of recurrence, based upon the statistical predictability of recurrence among pN0(mol+) patients compared to pN0(mol−).

### SUMMARY OF THE INVENTION

One aspect of the present invention provides a database for predicting clinical outcomes based upon quantitative tumor burden in lymph node samples from an individual. The database comprises data sets from a plurality of individuals. The data sets include clinical outcome data and data regarding number of lymph nodes evaluated, maximum number of biomarker detected in any single node, median normalized expression levels detected across all evaluated lymph nodes and the maximum normalized expression levels detected in any evaluated lymph nodes. The database also providing stratified risk categories based upon recursive partitioning of data.

Another aspect of the invention provides a system for predicting clinical outcomes based upon quantitative tumor burden in lymph node samples from an individual. The system comprises a database as set forth above. In addition, the system includes a data processor, an input interface and an output interface. The input interface allows for the input a test patient data set including data regarding number of lymph nodes evaluated, maximum number of biomarker detected in any single node, median normalized expression levels detected across all evaluated lymph nodes and the maximum normalized expression levels detected in any evaluated lymph nodes into the data processor which is linked to the data base. The data processor processes the inputted patient data with data in database and the test patient data is assigned to a stratified risk category. The output interface displays test patients identity and assigned stratified risk category.

Another aspect of the invention relates to a method of preparing a database as set forth above. The method comprises compiling data sets for a plurality of individuals which include clinical outcome data and data regarding number of lymph nodes evaluated, the maximum number of biomarker detected in any single node, median normalized expression levels detected across all evaluated lymph nodes and the maximum normalized expression levels detected in any evaluated lymph node. In addition, the data sets are processed using recursive partitioning to

Another aspect of the invention relates to a method for predicting clinical outcome for a test patient based upon quantitative tumor burden in lymph node samples from an individual. The method comprises measuring quantitative tumor burden in a plurality of lymph node samples from an individual. The quantitative tumor burden measurement data is inputted into the system set forth above and processing with data in in the database of the system. The results of the processing of the data is the assignment of data test patient to a stratified risk category. Output is produced that displays test patient's identity and assigned stratified risk category.

### DESCRIPTION OF THE FIGURES

FIG. 1 is a diagram of recursive partitioning of patients into risk strata based on the maximum copy number of GUCY2C in any node, the median normalized GUCY2C expression across all lymph nodes, and the maximum normalized expression of GUCY2C in any lymph node. Values represent the number of patients with recurrences/number of patients in strata.

FIG. 2 is data from Example 1 showing time to recurrence within risk strata defined by tumor burden quantified by recursive partitioning of GUCY2C expression.

FIG. 3 illustrates patient selection for analysis in Example 2.

FIG. 4 refers to data from Example 2. Time to recurrence (A) and disease-free survival (B) in patients with pN0 colorectal cancer stratified by recursive partitioning. Tables below Kaplan-Meier plots summarize the number of patients at risk as well as cumulative events for each outcome. Censored values in time to recurrence reflect death from another cancer, a noncancer-related death, and death because of the cancer treatment, or loss of follow-up of individual patients. Censored patients in disease-free survival reflect loss to follow-up.

FIG. 5 refers to data from Example 2. Time to recurrence (A) and disease-free survival (B) in patients with pN0 colon cancer stratified by recursive partitioning. Tables below Kaplan-Meier plots summarize the number of patients at risk as well as cumulative events for each outcome. Censored values in time to recurrence reflect death from another cancer, a noncancer-related death, and death because of the cancer treatment, or loss of follow-up of individual patients. Censored patients in disease-free survival reflect loss to follow-up.

FIG. 6 refers to data from Example 2. Time to recurrence (A, B) and disease-free survival (C, D) in patients with stage I (A, C) or II (B, D) colorectal cancer stratified by recursive partitioning. Tables below Kaplan-Meier plots summarize the number of patients at risk as well as cumulative events for each outcome. Censored values in time to recurrence reflect death from another cancer, a noncancer-related death, and death because of the cancer treatment, or loss of follow-up of individual patients. Censored patients in disease-free survival reflect loss to follow-up (22). For the analysis of time to recurrence, there were only 3 stage I patients stratified as pN0 (mol_{High}). At 6 months, one developed recurrence, one continues to be followed, and one was lost to follow-up.

FIG. 7 refers to data from Example 2. Cox proportional hazards analyses of time to recurrence in patients with pN0 colorectal cancer stratified by recursive partitioning. HRs (circles) with 95% C1s (horizontal lines) and P values for multivariable analyses describe interactions between prognostic characteristics and time to recurrence. Parameters that are significantly prognostic (P<0.05) are highlighted in red.

FIG. 8 refers to data from Example 2. Multivariable analyses employing Cox proportional hazards models were performed.

FIG. 9 refers to data from Example 3. Time to recurrence in patients with pN0 colorectal cancer stratified by occult tumor burden. Table summarizes the number of patients at risk as well as cumulative events for each outcome.

FIG. 10 refers to data from Example 3. Occult tumor burden in black and white patients. (A) Occult tumor cells in lymph nodes quantified by GUCY2C RT-PCR. Least squares mean and 95% confidence interval of relative GUYC2C expression in lymph nodes³⁴ in blacks and whites. In linear mixed effects model, with random patient effect, controlling for center to center differences, blacks have significantly higher levels of occult tumor cells in lymph nodes (p<0.001). (B) Stratification of prognostic risk by occult tumor burden in blacks and whites. Blacks are significantly more likely to be at high risk for disease recurrence based on occult tumor burden in lymph nodes (p=0.007).

FIG. 11 refers to data from Example 3. Distribution of black and white pN0 colorectal cancer patients with tumors with different T stages or lymph node collections stratified by occult tumor burden. Blacks are significantly more likely to be at high risk (p=0.007) versus low risk for disease recurrence based on occult tumor burden in lymph nodes regardless of T stage (p=0.006) or number of lymph nodes collected (p=0.02). P values reported from multivariate polytomous regression model for High vs Low Risk comparisons.

FIG. 12 refers to data from Example 4 showing time to recurrence plotting with risk of disease recurrence.

FIG. 13 refers to data from Example 4 showing predicted probability and risk level.

### DETAILED DESCRIPTION OF PREFERRED EMBODIMENTS

Quantitative methodologies for directly counting tumor cells in samples or by measuring the quantity of biomarkers associated with tumor cells provides powerful technology to measure tumor levels in lymph node samples. PCT application PCT/US09/043,857 and corresponding U.S. Published Patent Application US 2011/0195415 disclose efficient and effective methods of performing quantitative RT-PCR using an efficiency adjusting methodology which provides consistency among multiple quantitative RT-PCR assays. The quantitative RT-PCR assay disclosed therein is used to detect micrometastasis in lymph nodes of cancer patents deemed free of lymph node metastatis (pN0) by standard pathology methods. These pN0 patients may be identified a pN0mol+ using the efficiency adjusted quantitative RT-PCR assays cancer described therein for example. Quantitative methodologies such as the efficiency adjusted quantitative RT-PCR assays may be used to provide prognostic stratification based upon assesse tumor burden in lymph nodes of cancer patients.

Prognostic stratification can be achieved by methods in which tumor burden is quantitatively assessed based upon multiple lymph node samples. Data including for example the number of nodes evaluated and the quantity of tumor cells per sample such as the tumor cell quantity as assessed by the presence and quantity of a marker associated with tumor cells. Other data may include demographic and clinicopathologic data.

Tumor burden assessment may include maximum biomarker copy in any node, median expression across all nodes assessed and the maximum normalized expression in any node. Other values may include direct tumor cell counts per node, median number of tumor cells per node assessed and the maximum normalized number of tumor cells in any node. Other values may include average expression across all nodes assessed, percent nodes having greater than a threshold level of marker or direct quantified tumor cells, number/percent of nodes identified as negative for marker/tumor cells or below threshold.

The accumulation of data and corresponding outcomes associated with the various patients for whom individual data has been collected provides for a database of information which can be used to predict outcomes based upon a new patent data set. The database sets the parameters for stratifying risk and a new patient's data compared to the database allows for a prognosis to be formulated based upon mathematically processing information in the database to generate a predicted outcome.

As additional data is collected and can be correlated with outcome, including treatment, the database can be further refined and expanded. Increased numbers of data sets which include outcomes can be used to improve the more precisely determine risk stratification levels. The data, when correlated to therapeutic intervention and outcome also provides prognostic value in identifying and determining patient populations based upon tumor burden data that or likely or unlikely to benefit from various therapeutic strategies. Thus, a patient having undergone evaluation of tumor burden can using stratified risk assessments make therapeutic choices based upon more precise prognostic statistical data.

A database is provided for predicting clinical outcomes based upon quantitative tumor burden in lymph node samples from an individual. The database comprises data sets from a plurality of individuals. The data sets include clinical outcome data and data regarding number of lymph nodes evaluated, maximum number of biomarker detected in any single node, median normalized expression levels detected across all evaluated lymph nodes and the maximum normalized expression levels detected in any evaluated lymph nodes. Other data may also be included as discussed herein. The database also providing stratified risk categories based upon recursive partitioning of data. In some embodiments, the quantitative tumor burden is assessed by RT-PCR. In some embodiments, the quantitative tumor burden is determined by quantifying the biomarker GCC or a nucleic acid sequence molecule encoding GCC.

A system is provided for predicting clinical outcomes based upon quantitative tumor burden in lymph node samples from an individual. The system comprises a database linked to a data processor, an input interface and an output interface. The input interface allows for the input a test patient data set including data regarding number of lymph nodes evaluated, maximum number of biomarker detected in any single node, median normalized expression levels detected across all evaluated lymph nodes and the maximum normalized expression levels detected in any evaluated lymph nodes into the data processor which is linked to the data base. The data processor processes the inputted patient data with data in database and the test patient data is assigned to a stratified risk category. The output interface displays test patients identity and assigned stratified risk category. The input interface may be a data port linked to an automated quantitative detector used to determine tumor burden in a sample. In some embodiments, the input interface is a key pad for entering data generated with respect to tumor burden in a sample. The output interface may be a data port to another system which can display or generate reports that display results. The output interface may comprise a printer which prints a report containing test patient identity information and assigned stratified risk category. The output interface may comprise an electronic data generator which generates an electronic report containing test patient identity information and assigned stratified risk category.

Methods are provided for preparing a database. The method comprises compiling data sets for a plurality of individuals which include clinical outcome data and data regarding number of lymph nodes evaluated, the maximum number of biomarker detected in any single node, median normalized expression levels detected across all evaluated lymph nodes and the maximum normalized expression levels detected in any evaluated lymph node. Other data may also be included. The data sets are processed using recursive partitioning to produce stratified risk categories. In some embodiments, the method for preparing a database comprises processing data sets using recursive partitioning to produce stratified risk categories by first partitioning data sets based upon maximum copies on any node wherein data sets are divided into a high group and a low group; then partitioning data sets in said high group and said low group into four groups based upon median normalized expression levels detected across all evaluated lymph nodes to divide said high group into a high low group and a high-high group and to divide said low group into a low-low group and a low-high group; then partitioning data sets in said high-high group and said low-high group into four groups based upon maximum normalized expression levels detected in any evaluated lymph nodes to divide said high-high group into a high-high-high group and a high-high-low group and to divide said low-high group into a low-high-low group and a low-high-high group. The results generated are data sets divided into six groups total, 1) high-low, 2) high-high-low, 3) high-high-high, 4) low-low, 5) low-high-high, and 6) low-high-low. The outcomes associated with each data set in each group may be compared to the partitioned groups and used to determine risk categories. For example, 1) high-low, 2) high-high-low, and 4) low-low may be deemed low risk; 5) low-high-high may be deemed high risk and 3) high-high-high and 6) low-high-low may be independently assigned low, medium or high based upon outcome. In some embodiments, 1) high-low, 2) high-high-low, 4) low-low and 6) low-high-low are low risk; and 3) high-high-high and 5) low-high-high are high risk.

Another aspect of the invention relates to a method for predicting clinical outcome for a test patient based upon quantitative tumor burden in lymph node samples from an individual. The method comprises measuring quantitative tumor burden in a plurality of lymph node samples from an individual. The quantitative tumor burden measurement data is inputted into the system set forth above and processing with data in in the database of the system. The results of the processing of the data is the assignment of data test patient to a stratified risk category. Output is produced that displays test patient's identity and assigned stratified risk category.

The use of quantitative tumor burden data from lymph node samples is shown herein to provide highly reliable risk stratification. Generally, quantitative tumor burden data from a patient is most effective in the methods if the data is generated from greater than 10-15 nodes, i.e. >10, >11, >12, >13, >14 or >15 nodes. Predictive value increases further with greater number of nodes surveyed and used to generate data.

The quantitative level of occult tumor burden in regional lymph nodes, particular when measured from samples of multiple lymph nodes from an individual, provides the basis to stratify risk and provide a more precise and accurate prognosis. Stratification of risk within the pN0(mol+) group is particularly useful in assessing the risks and benefits of wait and see versus taking value of treatment options. Moreover, the quantitative level of occult tumor burden in regional lymph nodes, particular when measured from samples of multiple lymph nodes from an individual, provides the basis to stratify risk, particularly among specific individuals within the pN0(mol+) group. Accordingly, the quantitative measure of occult tumor burden levels in regional lymph nodes provides an improved prognostic indicator of likelihood or recurrence, allowing for more individualized decision making related to treatment options and determination of acceptable risk levels associated treatment side effects and toxicities. As part of a method of treating cancer, the improved prognostic determination provides improved methods of treating cancer. Additionally, the quantitative measure of occult tumor burden levels in regional lymph nodes provides an improved indicator of likelihood of response to therapeutic intervention, providing for improved evaluation of treatment options and treatment of cancer.

RT PCR offers a useful technique for detecting occult tumor cells in lymph nodes. In breast and other cancers, the categorical (yes/no) identification of micrometastases is clinically relevant. However, because of exquisite sensitivity, RT PCR can detect cancer cells in lymph nodes below the threshold of prognostic risk. Quantitative RT PCR (qRT PCR) offers an opportunity to enumerate tumor cells in lymph nodes and determine the relationship between variable tumor burden and disease risk. In addition, qRT PCR quantifies tumor cells in entire resection specimens. Thus, qRT PCR presents a previously unrecognized method to quantify molecular tumor burden across the regional lymph node network, providing an enhancement over current 2 dimensional histopathology estimates of tumor.

Accordingly, in some embodiments, the methods include the steps of detecting the level of biomarker mRNA present in lymph node sample using quantitative qRT-PCR comprising the steps of: isolating mRNA from lymph node samples obtained from an individual who has been diagnosed with cancer; performing qRT-PCR on at least a sample of the mRNA using the primers that amplify the biomarker; performing qRT-PCR on at least a sample of the mRNA using the primers that amplify a reference marker; and estimating by logistic regression analysis of amplification profiles from the qRT-PCR reactions to provide an efficiency-adjusted relative quantification based on parameter estimates from fitted models. Preferably, samples from multiple lymph nodes are evaluated. In some embodiments, the methods may further comprise comparing the efficiency-adjusted relative quantification to an established cut off. In some embodiments the efficiency-adjusted relative quantification is used to determine if the lymph node samples contains biomarker mRNA indicative of occult metastasis and the quantity of such biomarker mRNA as an indicator of occult metastasis tumor load. In some embodiments, the established cut off is the median of efficiency-adjusted relative quantifications compiled from a plurality of samples from a plurality of individuals. In some embodiments, the reference marker is beta actin. In some embodiments, a system comprises a device programmed to quantify biomarker mRNA by qRT-PCR in a sample using logistic regression analysis of amplification profiles from qRT-PCR reactions to produce an efficiency-adjusted relative quantification based on parameter estimates from fitted models. The device may be programmed to compare an efficiency-adjusted relative quantification with established cut off points in order to determine if a sample that was used to produce the efficiency-adjusted relative quantification contained a level of biomarker mRNA exceeding a specific threshold.

Quantitative measures of tumor burden include, for example, median biomarker mRNA copy number per lymph node, maximum biomarker mRNA copy number per lymph node, median relative biomarker mRNA expression per lymph node, maximum relative biomarker mRNA expression per lymph node, total biomarker mRNA copy number across all lymph nodes, and total relative biomarker mRNA expression across all lymph nodes, and the total number of lymph nodes positive for the biomarker mRNA. Quantitative measures of tumor burden may also include, for example, median biomarker protein copy number per lymph node, maximum biomarker protein copy number per lymph node, median relative biomarker protein expression per lymph node, maximum relative biomarker protein expression per lymph node, total biomarker protein copy number across all lymph nodes, and total relative biomarker protein expression across all lymph nodes, and the total number of lymph nodes positive for the biomarker protein. Quantitative measures of tumor burden may also include, for example, median cancer cell number per lymph node, maximum cancer cell number per lymph node, median relative cancer cell number per lymph node, maximum relative cancer cell number per lymph node, total cancer cell number across all lymph node, and total relative cancer cell across all lymph nodes, and the total number of lymph nodes positive for cancer cells. In each case, quantitative measure is elevated levels and/or above background and/or noise levels. Other variables for risk stratification may include known demographic factors such as age, gender, race, behavior factors such as smoking, substance abuse and dependency, family history and genetic factors, and clinicopathologic factors.

Quantitative level of occult tumor burden may also be measured by any of the several known methods of measuring tumor levels. Molecular pathology provides several options for quantitative assessment of tumor burden in lymph node samples. Direct counting of cancer cells such through the use of cell sorting based upon tumor marker expression may be carried out. Similarly, detection of levels of expression of markers can also be undertaken as such expression levels generally have some correlation to tumor cell number. Expression may be detected as protein levels or as mRNA levels. Techniques such as qRT-PCR disclosed above, branched oligonucleotide technology, Panomics QuantiGene® 2.0 (Affymetrix, Inc. Santa Clara, Calif.) Quantitative Gene expression reagents and assays, MassARRAY® (Sequenom, Inc. San Diego, Calif.) Quantitative Gene Expression systems in situ hybridization using detectable probes (such as FISH), dot blots assays, and other RNA quantitative amplification techniques and Northern Blots are useful for measuring mRNA levels and protein mass spectrometry including protein and peptide fractionation coupled with mass spectrometry, immunohistochemistry using detectable binding agents, immunoassays such as ELISA or Western blots, QProteome FFPE Qiagen Valencia Calif., reverse phase protein microarrays are useful for detecting protein markers presence and levels.

Cancers for which biomarkers are available which can be used to quantify tumor burden in a lymph node sample may be used. While not intending to be limited to the recited cancers, the most prevalent forms of cancer include Bladder, Breast Colon and Rectal, Endometrial, Kidney (Renal Cell) Cancer, Leukemia (All Types), Lung (Including Bronchus), Melanoma and other skin cancers, Non-Hodgkin Lymphoma, Pancreatic, Prostate and Thyroid. Cancers of the penis, vulva, cervix, head and neck (including brain, mouth, nasopharengeal, esophageal, larynx and throat), stomach, bone, and ovarian are also common.

Biomarkers include any moiety which if present on a cancer cell in the lymph node can be detected above any background associated with the detection technology and normal lymph node expression levels. In some embodiments, biomarkers which are not expressed in normal lymph node are preferred. In some embodiments, biomarkers which are expressed in a tissue specific manner or which are expressed in association with cancer (such as oncogenes and splice variants for example) are preferred. In some embodiments, biomarkers are detected as proteins or nucleic acid molecules which encode such proteins.

The intestinal tumor suppressor GUCY2C (guanylyl cyclase C or GCC) is the receptor for the paracrine hormones guanylin and uroguanylin, gene products universally lost early in intestinal neoplasia. Loss of hormone expression silences GUCY2C signaling which contributes to transformation by promoting proliferation, crypt hypertrophy, metabolic remodeling, and genomic instability. The highly selective expression by intestinal epithelial cells normally and universal overexpression by intestinal tumor cells make GUCY2C a candidate for a specific molecular marker for metastatic colorectal cancer. A recent prospective analysis revealed that pN0 colorectal cancer patients whose nodes were GUCY2C positive by molecular analysis suffered recurrence more frequently than those who had GUCY2C negative nodes (20% vs. 6%). Other cancer biomarkers according to some embodiments include GCC, alpha-Fetoprotein/AFP, ErbB2/Her2, CA125/MUC16, Kallikrein 3, PSA, ER alpha/NR3A1, Progesterone R/NR3C3, and ER beta/NR3A2, Progesterone R B/NR3C3, and EGFR mutant. In some embodiments, cancer biomarkers may be 5T4, M-CSF, 15-PGDH/HPGD, Matriptase/ST14, A33, MCAM/CD146, ABCB5, Mesothelin, ACE/CD143, Methionine Aminopeptidase, AG-2, Methionine Aminopeptidase 2/METAP2, AG-3, MIA, Annexin A3, MIF, APC, Mindin, Aurora A, MMP-2, beta-Catenin, MMP-3, BAP1, MMP-9, Bc1-2, Musashi-1 BMI-1, c-Myc, BRCA1, NCAM-L1/L1CAM, BRCA2, NDRG1, Brk, NEK2, BSRP-A, NELL1, c-Abl, NELL2, C4.4A/LYPD3, Nestin, Cadherin-13, NG2/MCSP, E-Cadherin, NKX3.1, Calretinin, Osteopontin/OPN, Carbonic Anhydrase IX/CA9, p21/CIP1/CDKN1A, Cathepsin D, p27/Kip1, Caveolin-2, p53, CCK4, p130Cas, CCR7, p15INK4b/CDKN2B, CCR9, p16INK4a/CDKN2A, CD24, PDCD4, CD31/PECAM-1, PDGF R beta, CD38, Peptidase Inhibitor 16/PI16, CD44, PGCP, CD63, PIWIL2, CD74, PLRP1, CD96, PRMT1, CD98, Prolactin, CD109, PSMA/FOLH1/NAALADase I, CDC73, PSP94/MSMB, CDX2, PTEN, CEACAM-4, PTH1R/PTHR1, CEACAM-5/CD66e, RAB25, CEACAM-6/CD66c, RARRES1, CEACAM-7, RARRES3, CEACAM-8/CD66b, Reg4, CHD1L, Ret, Chorionic Gonadotropin, alpha Chain (alpha HCG), RNF2, Cornulin, S100A1, Cortactin, S100A2, CTCF, S100A4, CXCL17/VCC-1, S100A6, CXCR4, S100A7, Cyclin D2, S100A16, DC-LAMP, S100B, DCBLD2/ESDN, S100P, DMBT1, SCF R/c-kit, DNMT1, Secretin R, DPPA4, Serpin A9/Centerin, ECM-1, Serpin E1/PAI-1, EGF, Serum Amyloid A4, EGF R/ErbB1, SEZ6L, ELF3, Skp2, EMMPRIN, SMAGP, EpCAM/TROP1, SOCS-1, ErbB3/Her3, SOCS-2, ErbB4/Her4, SOCS-6, ERK1, Soggy-1/DkkL1, FGF acidic, SOX2, FGF basic, Src, FGF R3, Stathmin/STMN1, Fibroblast Activation Protein alpha/FAP, STEAP1, FOLR1, STYK1, FOLR2, Survivin, FOLR3, Syndecan-1/CD138, FOLR4, Synuclein-gamma, FosB/GOS3, TCL1A, FoxO3, TCL1B, Galectin-3, TEM7/PLXDC1, Gastrokine 1, TEM8/ANTXR1, Glypican 3, TGF-beta 1, GRP78/HSPA5, TGF-beta 1, 2, 3, HE4/WFDC2, TGF-beta 1/1.2, Hepsin, TGF-beta 2/1.2, HGF R/c-MET, TGF-beta RI/ALK-5, HIN-1/SCGB3A1, THRSP, IGF-I, Thymosin beta 4, IGF-I R, Thymosin beta 10, IGF-II, TIMP, IGFBP-3, TIMP-1, IGFL-3, TIMP-2, IL-6, TIMP-3, ING1, TIMP-4, ITM2C, TLE1, JunB, TMEFF2/Tomoregulin-2, JunD, TNF-alpha/TNFSF1A, Kallikrein 2, TRA-1-85, Kallikrein 6/Neurosin, TRAF-4, KLF10 beta-III, Tubulin, KLF17, u-Plasminogen Activator/Urokinase, Leptin/OB, UBE2S, LKB1, uPAR, LRMP, VCAM-1/CD106, LRP-1B, VEGF, LRRC4, VEGF/P1GF Heterodimer, LRRN1/NLRR-1, VSIG1, LRRN3/NLRR-3, VSIG3, Ly6K, ZAG, LYPD1 and ZAP70.

The predictive value of quantitative level of occult tumor burden in regional lymph nodes is improved with the number of lymph node samples from an individual that qualitative measurements can be made. The number of lymph node samples may range from 2-200 or more. In some embodiments, the quantitative level of occult tumor burden is measured in samples of one, two, three, four, five, six, seven, eight, nine, ten, eleven or twelve different lymph nodes. In some embodiments, the quantitative level of occult tumor burden is measured in samples of twelve, thirteen, fourteen, fifteen, sixteen, seventeen, eighteen, nineteen, twenty, twenty one, twenty two twenty three or twenty four different lymph nodes. In some embodiments, the quantitative level of occult tumor burden is measured in samples of twenty three, twenty four, twenty five, twenty six, twenty seven, twenty eight, twenty nine, thirty, thirty one or thirty two different lymph nodes. In some embodiments, the quantitative level of occult tumor burden is measured in samples of forty three, forty four, forty five, forty six, forty seven, forty eight, forty nine, fifty, fifty one or fifty two or more different lymph nodes. In some embodiments, the quantitative level of occult tumor burden is measured in samples of more than fifty, more than sixty, more than seventy, more than eighty, more than ninety, more than one hundred, more than one hundred, more than one hundred, more than one hundred, more than one hundred ten, more than one hundred twenty, more than one hundred thirty, more than one hundred forty, more than one hundred fifty, more than one hundred sixty, more than one hundred seventy, more than one hundred eighty, more than one hundred ninety, or more than two hundred. In some embodiments, the quantitative level of occult tumor burden is measured in samples of 10-30 lymph nodes, 10 29 lymph nodes, 10-28 lymph nodes, 10-27 lymph nodes, 10-26 lymph nodes, 10-25 lymph nodes, 10-24 lymph nodes, 10-23 lymph nodes, 10-22 lymph nodes, 10-21 lymph nodes, 10-20 lymph nodes, 10-19 lymph nodes, 10-18 lymph nodes, 10-17 lymph nodes, 10-16 lymph nodes, 10-15 lymph nodes, 10-14 lymph nodes, 10-13 lymph nodes, 10-12 lymph nodes, 10 or 11 lymph nodes, 11-30 lymph nodes, 11 29 lymph nodes, 11-28 lymph nodes, 11-27 lymph nodes, 11-26 lymph nodes, 11-25 lymph nodes, 11-24 lymph nodes, 11-23 lymph nodes, 11-22 lymph nodes, 11-21 lymph nodes, 11-20 lymph nodes, 11-19 lymph nodes, 11-18 lymph nodes, 11-17 lymph nodes, 11-16 lymph nodes, 11-15 lymph nodes, 11-14 lymph nodes, 11-13 lymph nodes, 11 or 12 lymph nodes, 12-30 lymph nodes, 12-29 lymph nodes, 12-28 lymph nodes, 12-27 lymph nodes, 12-26 lymph nodes, 12-25 lymph nodes, 12-24 lymph nodes, 12-23 lymph nodes, 12-22 lymph nodes, 12-21 lymph nodes, 12-20 lymph nodes, 12-19 lymph nodes, 12-18 lymph nodes, 12-17 lymph nodes, 12-16 lymph nodes, 12-15 lymph nodes, 12-14 lymph nodes, 12 or 13 lymph nodes, 13-30 lymph nodes, 13-29 lymph nodes, 13-28 lymph nodes, 13-27 lymph nodes, 13-26 lymph nodes, 13-25 lymph nodes, 13-24 lymph nodes, 13-23 lymph nodes, 13-22 lymph nodes, 13-21 lymph nodes, 13-20 lymph nodes, 13-19 lymph nodes, 13-18 lymph nodes, 13-17 lymph nodes, 13-16 lymph nodes, 13-15 lymph nodes, 13 or 14 lymph nodes, 14-30 lymph nodes, 14-29 lymph nodes, 14-28 lymph nodes, 14-27 lymph nodes, 14-26 lymph nodes, 14-25 lymph nodes, 14-24 lymph nodes, 14-23 lymph nodes, 14-22 lymph nodes, 14-21 lymph nodes, 14-20 lymph nodes, 14-19 lymph nodes, 14-18 lymph nodes, 14-17 lymph nodes, 14-16 lymph nodes, 14 or 15 lymph nodes, 15-30 lymph nodes, 15-29 lymph nodes, 15-28 lymph nodes, 15-27 lymph nodes, 15-26 lymph nodes, 15-25 lymph nodes, 15-24 lymph nodes, 15-23 lymph nodes, 15-22 lymph nodes, 15-21 lymph nodes, 15-20 lymph nodes, 15-19 lymph nodes, 15-18 lymph nodes, 15-17 lymph nodes, 15 or 16 lymph nodes, 16-30 lymph nodes, 16-29 lymph nodes, 16-28 lymph nodes, 16-27 lymph nodes, 16-26 lymph nodes, 16-25 lymph nodes, 16-24 lymph nodes, 16-23 lymph nodes, 16-22 lymph nodes, 16-21 lymph nodes, 16-20 lymph nodes, 16-19 lymph nodes, 16-18 lymph nodes, 16 or 17 lymph nodes, 17-30 lymph nodes, 17-29 lymph nodes, 17-28 lymph nodes, 17-27 lymph nodes, 17-26 lymph nodes, 17-25 lymph nodes, 17-24 lymph nodes, 17-23 lymph nodes, 17-22 lymph nodes, 17-21 lymph nodes, 17-20 lymph nodes, 17-19 lymph nodes, 17 or 18 lymph nodes, 18-30 lymph nodes, 18-29 lymph nodes, 18-28 lymph nodes, 18-27 lymph nodes, 18-26 lymph nodes, 18-25 lymph nodes, 18-24 lymph nodes, 18-23 lymph nodes, 18-22 lymph nodes, 18-21 lymph nodes, 18-20 lymph nodes, 18 or 19 lymph nodes, 19-30 lymph nodes, 19-29 lymph nodes, 19-28 lymph nodes, 19-27 lymph nodes, 19-26 lymph nodes, 19-25 lymph nodes, 19-24 lymph nodes, 19-23 lymph nodes, 19-22 lymph nodes, 19-21 lymph nodes, 19 or 20 lymph nodes, 20-30 lymph nodes, 20-29 lymph nodes, 20-28 lymph nodes, 20-27 lymph nodes, 20-26 lymph nodes, 20-25 lymph nodes, 20-24 lymph nodes, 20-23 lymph nodes, 20-22 lymph nodes, 20 or 21 lymph nodes, 21-30 lymph nodes, 21-29 lymph nodes, 21-28 lymph nodes, 21-27 lymph nodes, 21-26 lymph nodes, 21-25 lymph nodes, 21-24 lymph nodes, 21-23 lymph nodes, 21 or 22 lymph nodes, 22-30 lymph nodes, 22-29 lymph nodes, 22-28 lymph nodes, 22-27 lymph nodes, 22-26 lymph nodes, 22-25 lymph nodes, 22-24 lymph nodes, 22 or 23 lymph nodes, 23-30 lymph nodes, 23-29 lymph nodes, 23-28 lymph nodes, 23-27 lymph nodes, 23-26 lymph nodes, 23-25 lymph nodes, 23 or 24 lymph nodes, 24-30 lymph nodes, 24-29 lymph nodes, 24-28 lymph nodes, 24-27 lymph nodes, 24-26 lymph nodes, 24 or 25 lymph nodes, 25-30 lymph nodes, 25-29 lymph nodes, 25-28 lymph nodes, 25-27 lymph nodes, 25 or 26 lymph nodes, 27-30 lymph nodes, 27-29 lymph nodes, 27 or 28 lymph nodes, 28-30 lymph nodes, 28 or 29 lymph nodes, 29-30 lymph nodes,

Once the quantitative level of occult tumor burden is measured to provide one or more quantitative measures of tumor burden described above (i.e, for example, median biomarker mRNA copy number per lymph node, maximum biomarker mRNA copy number per lymph node, median relative biomarker mRNA expression per lymph node, maximum relative biomarker mRNA expression per lymph node, total biomarker mRNA copy number across all lymph nodes, and total relative biomarker mRNA expression across all lymph nodes, and the total number of lymph nodes positive for the biomarker mRNA in a particular number of lymph nodes), the data is compared to a database which includes quantitative level of occult tumor burden is measured in numerous different numbers of lymph nodes and the respective outcome in each instance. The database is a compilation of previous measurements and outcomes, particularly incidence of recurrence/disease free, time of recurrence, survival over 1, 2, 3, 4, 5, 6, 7, 8, 9, 10, 11, 12, 13, 14, 15, 16, 17, 18, 19, 20 and/or more years. The database may also optionally include the adjunctive therapeutic interventions performed as well as, optionally, demographic data such as age, gender, race, behavioral factors such as smoking, substance abuse and dependency, family history and genetic data, and/or clinicopathologic data.

The database preferably includes patient data with outcomes for at least 10 patients each for each number of lymph nodes included in the database. For example, if the database includes data for 1-40 lymphnodes, the database will contain data with outcomes from at least 10 patients who had quantitative measure of tumor burden in one lymph node, from at least 10 patients who had quantitative measure of tumor burden in two lymph nodes, from at least 10 patients who had quantitative measure of tumor burden in three lymph nodes, from at least 10 patients who had quantitative measure of tumor burden in four lymph nodes, from at least 10 patients who had quantitative measure of tumor burden in five lymph node, etc . . . through and to from at least 10 patients who had quantitative measure of tumor burden in forty lymph nodes. The database preferably includes patient data with outcomes for at least 20 patients each for each number of lymph nodes included in the database. The database preferably includes patient data with outcomes for at least 30 patients each for each number of lymph nodes included in the database. The database preferably includes patient data with outcomes for at least 40 patients each for each number of lymph nodes included in the database. The database preferably includes patient data with outcomes for at least 50 patients each for each number of lymph nodes included in the database. The database preferably includes patient data with outcomes for at least 60 patients each for each number of lymph nodes included in the database. The database preferably includes patient data with outcomes for at least 70 patients each for each number of lymph nodes included in the database. The database preferably includes patient data with outcomes for at least 80 patients each for each number of lymph nodes included in the database. The database preferably includes patient data with outcomes for at least 90 patients each for each number of lymph nodes included in the database. The database preferably includes patient data with outcomes for at least 40 patients each for each number of lymph nodes included in the database. The database preferably includes patient data with outcomes for at least 50 patients each for each number of lymph nodes included in the database. The database preferably includes patient data with outcomes for at least 100 or more patients each for each number of lymph nodes included in the database. The database is preferably designed to be dynamic so that it can be updated as additional data with outcomes is available. As more data with outcomes are collected and added to the database, the predictive value of the results becomes greater and greater and the patient data which correlates to particular risk level becomes more and more refined. In some embodiments, the database may be replaced with specific criteria for analyzing patient data based upon the refined data of the data base.

The database represent results of recursive portioning of data from previous patients with respect to tumor burden as defines by one or more quantitative measures of tumor burden, number of nodes tested and patient outcome including recurrence, time without recurrence/time to recurrence, life expectancy, death associated with recurrence, and/or death by cause other than recurrence of cancer. Quantitative measures of tumor burden data and various other input data together with the corresponding outcomes allow for the use of recursive partitioning to stratify and assess risk for a given outcome based upon factors including quantitative measures of tumor burden data.

Recursive partitioning is well known statistical method of multivariable analysis. Using recursive partitioning, data is grouped based upon the various known data of patients and their outcomes, the result being predictive values for risks or probability of an outcome for a given data set. Using quantitative measures of tumor burden data and corresponding data related to outcome, the statistical risk or probability of recurrence can be determined based upon quantitative measures of tumor burden and such risk/probability determination can be used to determine a patient's risk/probability of recurrence based upon that patient's quantitative measures of tumor burden data. While in some embodiments, data collection including quantitative measures of tumor burden data and outcomes can be compiled and used in recursive partitioning to determine the risk/probability of an outcome based upon quantitative measures of tumor burden of a patient, in some embodiments, a database is provided which contains the corresponding risk/probability of various outcomes based upon specific patient data including quantitative measure of tumor burden data and outcomes. The database may be part of a system in which specific patient data is inputted using a data function and that data is processed using the database in the system to identify risk/probability of various outcomes based upon the patient data provided. The system may rely upon previously calculated risk/probability determination and/or it may provide analysis of data based upon multiple combinations of factors.

The total occult tumor burden in regional lymphnodes may be correlated to outcome. As data is compiled the tumor burden quantity and number of tumors involved data becomes increasingly more precise in its predictive capacity. Initially, a patient's number of lymph nodes tested to number of lymphnodes deemed pN0(mol+) plus the quantity of tumor as represented by marker levels for example in each pN0(mol+) lymph node is compared to the database, particularly with respect to data from patients which had the same number of lymph nodes tested as the patient. In some embodiments, outcomes are grouped according to the distribution of total quantity of tumor among the pN0(mol+) nodes. Algorithms may be used to determine the weight the various factors such as number of pN0(mol+) nodes, quantity of tumor in each node, total quantity of tumor in all nodes, distribution of tumor among pN0(mol+) nodes. Using the data from individuals which includes outcomes, a predictive model is provided for which a patient's data may be compared.

The outcome grouping that the patient's data most closely resembles allows the risk/outcome likelihood to be assessed for the patient. In this way, pN0(mol+) patients can be stratified into one of several risk/outcome likelihood groupings. For example, in some embodiments, the database provides three groupings for pN0(mol+) patients: high risk, medium risk or low risk, based upon the evaluation of tumor burden data and outcome. Thus, for example, while pN0(mol+) patients may have a 35% chance of recurrence, high risk patients may actually have a 75-80% chance while low risk patient may have less than 5% chance with medium risk patients have risk levels between the two. Although a pN0, such a patient how is identified as a high risk pN0(mol+) would like choose a course of treatment that is more aggressive than one typically chosen by someone deemed pN0 or even pN0(mol+). Similarly, the low risk pN0(mol+) would likely consider the risk and side effects of adjunctive therapy disproportionately unacceptable. The technology that correlates quantity of occult tumor burden in regional lymphnodes to likely outcome provides powerful prognostic ability in the treatment of cancer patients relative to current conventional methods.

Systems may include kits for performing quantitative assays and an interface that allows for patient data to be entered after which is transmitted or otherwise compared to the database for comparison and determination of the patients risk group. Systems may include kits for performing quantitative assays and an interface that allows for patient data to be entered after which is transmitted to or otherwise delivered to a processing unit where the data is processed using an algorithm for example prior to comparison to data in the database. Databases may be included as part of the system and saved within the processing unit storage function or on portable data storage unit such as a CD-ROM, or the system may include components or information which provides access to the database which is maintained at a central location remote from the laboratory/hospital site.

Some methods of the invention comprise performing quantitative assays and transmitting data. Some methods of the invention comprise performing quantitative assays and inputting data using an interface which is capable of exchanging data with a processing unit and/or database. Some methods of the invention comprise performing quantitative assays and inputting data using an interface which is capable of exchanging data with a processing unit and/or database which uses inputted data to determine outcome risk/probability and communicate the same. Some methods of the invention comprise performing quantitative assays and inputting data using an interface which is capable of exchanging data with a processing unit and/or database which uses inputted data to determine outcome risk/probability and communicate the same via a user interface.

Methods, kits and systems are provided that can determine relative quantity of GCC mRNA in a sample or series of samples. These methods, kits and systems may be useful to detect metastasis in patients diagnosed with primary colorectal, gastric or esophageal cancer. These methods, kits and systems may be useful to detect metastasis in patients diagnosed with primary colorectal, gastric or esophageal cancer. These methods, kits and systems may be useful to screen individuals for metastatic colorectal, gastric or esophageal cancer. These methods, kits and systems may be useful to predict the risk of occurrence of relapse in patients diagnosed with primary colorectal, gastric or esophageal cancer.

Methods, kits and systems are provided for detecting the level of GCC encoding mRNA present in a sample using quantitative (q) RT-PCR.

In some aspects, the methods comprise the steps of: obtaining one or more tissue samples from an individual; isolating RNA from said sample; and performing quantitative RT-PCR using the primers ATTCTAGTGGATCTTTTCAATGACCA (SEQ ID NO:1) and CGTCAGAACAAG-GACATTTTTCAT (SEQ ID NO:2). In some embodiments, the methods further comprising using a Taqman probe (FAM-TACTTGGAGGACAATGTCACAG-CCCCTG-TAMRA) (SEQ ID NO:3) in the quantitative RT-PCR.

In some aspects of the invention, the methods comprise the steps of: obtaining one or more tissue samples from an individual; isolating RNA from said sample; performing quantitative RT-PCR using the primers that amplify GCC; and performing quantitative RT-PCR using the primers that amplify a reference marker such as beta-actin. In some embodiments the methods comprise performing quantitative RT-PCR using the primers that amplify GCC in which the primers are ATTCTAGTGGATCTTTTCAATGACCA (SEQ ID NO:1) and CGTCAGAACAAG-GACATTTTTCAT (SEQ ID NO:2). In some embodiments, the methods further comprising using a Taqman probe (FAM-TACTTGGAGGACAATGTCACAG-CCCCTG-TAMRA) (SEQ ID NO:3) in the quantitative RT-PCR. In some embodiments, the methods comprise performing quantitative RT-PCR using the primers that amplify beta-actin, in which the primers are CCACACTGTGCCCATCTACG (SEQ ID NO:4) and AGGATCTTCATGAG-GTAGTCAGTCAG (SEQ ID NO:5). In some embodiments, the methods further comprise using a Taqman probe(FAM-ATGCCC-X(TAMRA)-CCCCCATGCCATCCTGCGTp) (SEQ ID NO:6).

In some aspects of the invention, the methods comprise the steps of: obtaining one or more tissue samples from an individual, isolating RNA from said sample, performing quantitative RT-PCR to amplify GCC and a reference marker such as beta-actin, and efficiency adjusting quantitative RT-PCR data based on parameter estimates from fitted models. The efficiency adjusting relative quantity of GCC mRNA may be scored using a predetermined cut off for positive or negative results such as the median efficiency adjusting relative quantity of GCC mRNA in multiple samples from multiple patients. In some embodiments, quantitative RT-PCR to amplify GCC is performed using the primers ATTCTAGTGGATCTTTTCAATGACCA (SEQ ID NO:1) and CGTCAGAACAAG-GACATTTTTCAT (SEQ ID NO:2). In some embodiments, the methods further comprise using a Taqman probe (FAM-TACTTGGAGGACAATGTCACAG-CCCCTG-TAMRA) (SEQ ID NO:3) in the quantitative RT-PCR. In some embodiments, the reference marker is beta-actin and the methods further comprise performing quantitative RT-PCR using the primers that amplify beta-actin using primers CCACACTGTGCCCATCTACG (SEQ ID NO:4) and AGGATCTTCATGAG-GTAGTCAGTCAG (SEQ ID NO:5). In some embodiments, the methods further comprise using a Taqman probe (FAM-ATGCCC-X(TAMRA)-CCCCCATGCCATCCTGCGTp) (SEQ ID NO:6).

In some aspects of the invention, the methods utilize one or more samples from a patient diagnosed with primary colorectal, gastric or esophageal cancer. In some embodiments, the sample is a lymph node sample. In some embodiments, a plurality of lymph node samples are used including, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more samples obtained from the patient. In some aspects of the invention, the data from the methods may be used to determine risk of recurrence.

The present invention provides kits for amplifying GCC-encoding mRNA. The kits may comprise RT-PCR primers ATTCTAGTGGATCTTTTCAATGACCA (SEQ ID NO:1) and CGTCAGAACAAG-GACATTTTTCAT (SEQ ID NO:2). In some embodiments, the kits may further comprise Taqman probe (FAM-TACTTGGAGGACAATGTCACAG-CCCCTG-TAMRA) (SEQ ID NO:3). In some embodiments, the kits may further primers CCACACTGTGCCCATCTACG (SEQ ID NO:4) and AGGATCTTCATGAG-GTAGTCAGTCAG (SEQ ID NO:5). In some embodiments, the kits may further comprise Taqman probe (FAM-ATGCCC-X(TAMRA)-CCCCCATGCCATCCTGCGTp) (SEQ ID NO:6). In some embodiments, the kits may further comprise instructions for programming a device to calculate the relative quantity of GCC mRNA using efficiency adjusting quantitative RT-PCR data based on parameter estimates from fitted models. Such instructions may be copied to a fixed medium. In some embodiments, the kits may further comprise instructions for programming a device to score the results of qPCR samples based upon relative quantity of GCC mRNA using efficiency adjusting quantitative RT-PCR data based on parameter estimates from fitted models. Such scoring may use a predetermined cut off or the median of aggregated data. Such instructions may be fixed to a medium.

The present invention provides compositions for amplifying GCC-encoding mRNA. The compositions may comprise ATTCTAGTGGATCTTTTCAATGACCA (SEQ ID NO:1) and CGTCAGAACAAG-GACATTTTTCAT (SEQ ID NO:2). In some embodiments, the compositions may further comprise (FAM-ATGCCC-X(TAMRA)-CCCCCATGCCATCCTGCGTp) (SEQ ID NO:3).

In some embodiments, the compositions may further comprise CCACACTGTGCCCATCTACG (SEQ ID NO:4) and AGGATCTTCATGAG-GTAGTCAGTCAG (SEQ ID NO:5). In some embodiments, the compositions may further comprise Taqman probe (FAM-ATGCCC-X(TAMRA)-CCCCCATGCCATCCTGCGTp) (SEQ ID NO:6).

The present invention provides systems for quantifying GCC encoding mRNA by quantitative (q) RT-PCR comprising a device programmed to process quantitative RT-PCR data by efficiency adjusting quantitative RT-PCR data based on parameter estimates from fitted models.

The present invention provides systems for determining if a patient has metastatic colorectal, gastric or esophageal cancer by comprising a device programmed to process quantitative RT-PCR data by efficiency adjusting quantitative RT-PCR data based on parameter estimates from fitted models.

The present invention provides for determining risk of recurrence in a patient diagnosed with colorectal, gastric or esophageal cancer comprising a device programmed to process quantitative RT-PCR data by efficiency adjusting quantitative RT-PCR data based on parameter estimates from fitted models.

The methods, kits compositions and systems may also be adapted for determining whether a patient with esophageal dysplasia or otherwise abnormally appearing tissue has Barrett's esophagus. Quantitative RT-PCR amplifying GCC-encoding mRNA may be performed as described herein on esophageal tissue samples to detect GCC mRNA levels and determining whether the results indicate Barrett's esophagus.

One problem associated with the detection of a marker using amplification is the false positives caused by background amplification product. In addition, simple detection assays provide limited information with respect to the degree of marker present. Quantitative amplification such as quantitative PCR overcomes the problems associated with background and provides more information with respect to the degree of target transcript than a simple detection assay.

In addition to the amount of marker present in a sample, quantitative PCR results are affected by the integrity of the sample from the time it is obtained to the time the amplification is performed. Further, the efficiency of the PCR reaction can vary from one sample to another. Thus, when performing quantitative PCR on multiple samples, methods are provided herein to allow for adjusting results to yield relative quantification based results of qPCR of a reference marker such as beta-actin. The GCC qPCR data is adjusted relative to the beta actin qPCR data so that the resulting quantification reflects a relative level of GCC mRNA to reference marker. Accordingly, results can be compared between samples even if a sample has been compromised with respect to degradation or if the reaction performed on a given sample proceeds relatively inefficiently. The relative quantification thereby reduces or eliminates differences in results arising from differences in sample integrity and reaction efficiency among the several samples by producing an output which is normalized with respect to the output from other samples.

By performing quantitative PCR on a reference marker that is present in a sample, such as beta actin, together with performing quantitative PCR on the target marker, such as GCC, the quantitative results of GCC present in a sample can be adjusted and expressed as a relative quantification which corresponds to the number of copies of GCC mRNA as a function of its relationship to the quantity of reference marker. When performing individual quantitative PCR reactions on multiple samples for GCC and a reference marker, the adjustment of results for each sample by logistic regression analyses provides test results which have relative quantification with reduced bias and error. Thus, the results account for the difference in integrity of samples and efficiencies of reactions, yielding relative quantification that more closely reflects the relative amount of amplification target present in the samples.

The reference marker can be any transcript that is known to be present in a sample in an amount within known range. Housekeeping proteins such as beta-actin are useful as reference markers. Amplification of GCC and beta actin transcripts can be performed in a single sample using a multiplex PCR method or a sample can be divided and the reactions can be performed separately. The results of GCC quantification are adjusted based upon the results of the beta actin quantification. By performing beta actin amplifications with GCC amplifications for multiple samples and adjusting the GCC quantification with the beta actin quantification results from the same sample, the resulting output provides a relative quantification of GCC and all results are adjusted to the same standard, reducing or eliminating bias and error from the overall results.

Aspects of the invention relate to methods which include the steps of performing quantitative amplification reactions for GCC and a reference marker such as beta actin and normalizing the GCC results to those for the reference marker to yield a relative quantification of GCC. Each sample is normalized to the reference marker present in that sample to produce relative quantities of GCC with respect to quantities of reference marker. Each relative quantity of GCC determined for each sample can be compared to another other relative quantity of GCC determined for another sample and the comparison reflect the differences in quantification of one sample compared to another, regardless of any differences in sample integrity or reaction efficiencies.

Once relative quantification is determined for multiple samples, the scoring of a sample as positive or negative is achieved by establishing the cut off. One way to establish a cut off is to compile results from a large number of individuals. The median may be calculated and used as the threshold. Those samples in which the relative quantity of GCC is equal to or greater than the median may be scored as positive and those below may be scored as negative. The presence of one positive node can be used to establish an individual as mol+.

As described herein, the quantity of GCC is the relative quantity with respect to the quantity of beta actin rather than an absolute quantification. By calculating relative quantity to a reference marker, the data from all samples is normalized with respect to reference marker and thus to each other. This method removes the variability associated with sample integrity and reaction efficiency that may occur between different samples.

Alternatively, at the time samples are collected, they may be spiked with a known quantity of a reference marker, for example a non-human sequence. Amplification of GCC and the reference maker is performed and quantification results of GCC for may be normalized against the results for the spiked reference marker. It is also envisioned that, the sample may be spiked with a known quantity of a reference marker, for example a non-human sequence, immediately prior to amplification. Amplification of GCC and the reference maker is performed and quantification results of GCC for may be normalized against the results for the spiked reference marker. It is also envisioned that two reference markers may be used, one spiked at the time of collection and one immediately prior to amplification. Spike references may also be used in conjunction with endogenous reference markers.

Systems are provided which include data processing devices which are programmed to calculate relative quantification data by efficiency adjusting quantitative RT-PCR data based on parameter estimates from fitted models. Such devices may be programmed to calculate relative quantities of GCC based upon quantitative results for reference markers such as beta actin. In addition, such devices may be programmed to score results for samples based upon data collected from a plurality of samples. The programming instructions may be provided on a fixed medium which can be used to program a device. A copy of the fixed medium containing the programming instructions may be provided with kits such as those with a container comprising GCC qPCR primers, optionally containers comprising reference marker such as beta actin qPCR primers, optionally positive and/or negative controls and/or instructions for performing the methods.

### EXAMPLES

### Example 1

Although ostensibly rendered tumor-free by surgery, ˜25% of patients with lymph nodes devoid of colorectal cancer by histopathology (pN0) suffer recurrence, suggesting the presence of occult metastases. GUCY2C, an intestinal tumor suppressor universally silenced in neoplasia, is a mechanism-based biomarker for metastatic colorectal cancer cells. Here, we explored the novel hypothesis that occult tumor burden, in which the amount of molecular metastases was estimated by GUCY2C quantitative RT-PCR (qRT-PCR), establishes prognostic risk to accurately stage pN0 patients. We demonstrate for the first time that occult tumor burden assessed across the regional lymph node network is a powerful independent prognostic marker of time to recurrence and disease-free survival in pN0 patients. This approach can improve prognostic risk stratification and chemotherapeutic allocation in pN0 patients. More generally, this study reveals a previously unappreciated paradigm to advance cancer staging, clinically translating emerging molecular platforms that complement histopathology, laboratory diagnostic, and imaging modalities.

Regional lymph node metastasis is the single most important prognostic factor in patients with colorectal cancer. Although theoretically rendered cancer free by surgery, patients with nodes devoid of histopathologic evidence of cancer (pN0) suffer recurrence rates of approximately 25%, while those rates exceed 50% in patients with >4 lymph nodes harboring metastases (pN2). Adjuvant chemotherapy improves disease-free and overall survival in patients with histopathologically evident lymph node metastases, but its role in pN0 patients remains unclear.

Recurrence in a substantial fraction of node-negative colorectal cancer patients suggests the presence of occult metastases in regional lymph nodes that escape standard detection methods. Conversely, patients who are free of lymph node metastases by any detection method may have a better prognosis. Clinically, more accurate assessment of occult metastases would improve risk stratification in a clinically heterogeneous population where up to 25% of patients “cured” by standard care suffer recurrence. In addition, patients with occult metastases at elevated recurrence risk might benefit from the increasingly effective adjuvant chemotherapy available for colorectal cancer.

GUCY2C (guanylyl cyclase C) is the intestinal tumor suppressing receptor for the paracrine hormones guanylin and uroguanylin, gene products universally lost early in intestinal neoplasia. Loss of hormone expression and GUCY2C silencing contribute to neoplastic transformation through unrestricted proliferation, crypt hypertrophy, metabolic remodeling and genomic instability. Highly selective expression by intestinal epithelial cells normally, and universal over-expression by intestinal tumor cells, suggested that GUCY2C might be a specific molecular marker for metastatic colorectal cancer. A recent prospective analysis revealed that pN0 colorectal cancer patients whose nodes were GUCY2C positive by molecular analysis suffered recurrence more frequently than those who had GUCY2C-negative nodes (20% v. 6%).

RT-PCR offers a unique opportunity to detect occult tumor cells in lymph nodes. In breast and other cancers, the categorical (yes/no) identification of micrometastases is clinically relevant. However, due to exquisite sensitivity, RT-PCR can detect cancer cells in lymph nodes below the threshold of prognostic risk. Quantitative (q)RT-PCR offers an opportunity to enumerate tumor cells in lymph nodes and by extension, examine the relationship between variable tumor burden and disease risk. In addition, qRT-PCR quantifies tumor cells in entire resection specimens. Thus, qRT-PCR presents a previously unrecognized method to quantify molecular tumor burden across the regional lymph node network, providing an enhancement over current 2-dimensional histopathology estimates of tumor.

The present analysis defines the association between occult tumor burden in lymph nodes, estimated by GUCY2C qRT-PCR, and time to recurrence and disease-free survival in patients with pN0 colorectal cancer.

Metastatic tumor burden is measured utilizing disease-specific biomarkers by quantitative reverse transcription polymerase chain reaction (q-RT-PCR) in tissues, including but not limited to, lymph nodes from patients. The summary measures of these markers are then representative of the amount of tumor that has spread to tissues, including, but not limited to, disease that is undetectable by pathologist due to both sampling of tissue (viewing individual small slice of node) and limitations of assessment utilizing current techniques.

In our current study in colon cancer, this method detects in very early stage patients, the subsets of patients at much higher risk of recurrent disease. Since this can be detected at or near the time of diagnosis, appropriate decisions for therapeutic approaches can be made. In the current AJCC patient staging paradigm, all early stage patients receive surgery alone, and clinical trials have not been able to demonstrate sufficient benefit of treatment with chemotherapy in AJCC Stage I and II patients. The proposed algorithms could be used to personalize treatment selection in these patients.

Occult Tumor Burden Quantified by GUCY2C qRT-PCR Stratifies Risk in pN0 Colorectal Cancer.

Although a high proportion of pN0 patients exhibit occult metastases by GUCY2C qRT-PCR, most pN0 patients will not recur. Reconciliation of this apparent inconsistency relies on the recognition that the categorical (yes/no) presence of nodal metastases does not assure recurrence but, rather, indicates risk. Indeed, only ˜50% of stage III patients, all of whom have histologically-detectable nodal metastases, ultimately develop recurrent disease. There is an emerging paradigm that goes beyond the categorical (yes/no) presence of tumor cells, to quantify metastatic tumor burden (how much) to more accurately stratify risk. This is exemplified by the relationship between prognostic risk and number of lymph nodes harboring tumor cells by histology where stage III patients with ≧4 involved nodes exhibit a recurrence rate that is 50-100% greater than those with ≦3 involved nodes. In that context, the prognostic value of the number of lymph nodes harboring tumor cells by GUCY2C qRT-PCR suggests an analogous relationship between occult tumor burden and risk. Beyond the number of involved lymph nodes, there is a relationship between the volume of cancer cells in individual nodes and prognostic risk, and metastases≧2 mm are associated with increased disease recurrence while the relationship between individual tumor cells or nests<0.2 mm and risk remains undefined.

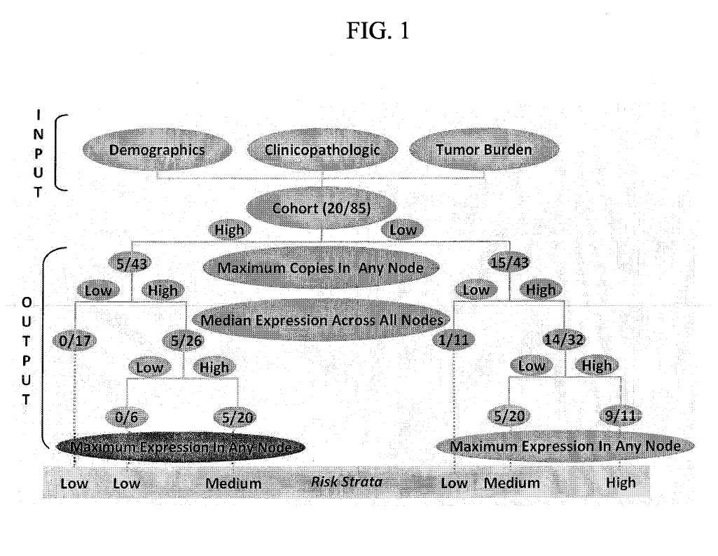

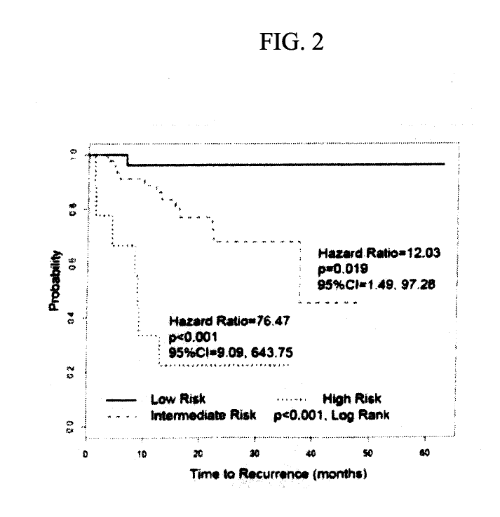

In that regard, one limitation to qualitative RT-PCR generally, and GUCY2C RT-PCR specifically, for categorical (yes/no) identification of occult metastases is the absence of information about tumor burden. Indeed, the superior sensitivity of qualitative RT-PCR, with its optimum tissue sampling and capacity for single cell discrimination, identifies occult metastases below the threshold of prognostic risk, limiting the specificity of molecular staging. However, the emergence of quantitative RT-PCR (qRT-PCR) provides an unprecedented opportunity to quantify occult tumor burden to assign prognostic risk, although this application has not been defined previously. Indeed, it remains unknown which quantitative parameters of tumor burden in lymph nodes estimated by qRT-PCR reflect risk and prognosis. In the absence of prior experience in the field, recursive partitioning was employed to objectively identify parameters that define homogeneous subgroups of prognostic risk in pN0 patients (FIG. 1). Here, recursive partitioning was applied to the pN0 population of patients with full collections of lymph nodes (>12; required for the analysis; n=85 patients), using all known prognostic demographic and clinicopathologic variables for risk stratification. In addition, quantitative measures of tumor burden established by GUCY2C qRT-PCR were used, including median copy number, maximum copy number, median relative expression, maximum relative expression, total copy number, and total relative expression across all lymph nodes, and the total number of lymph nodes positive by GUCY2C qRT-PCR. Unexpectedly, the algorithm selected only quantitative measures of tumor burden established by GUCY2C qRT-PCR, including maximum GUCY2C mRNA copy number in any lymph node, normalized median GUCY2C mRNA expression across all lymph nodes, and maximum normalized GUCY2C expression in any lymph node (FIG. 1). Integration of these quantitative measures of GUCY2C expression essentially provides a molecular analogue of morphological assessment of metastatic volumes in lymph nodes. Moreover, combining molecular detection and recursive partitioning augments 2-dimensional morphology by quantifying metastases in a large volume of tissue (the entire sample), rather than a single thin section, and across all available lymph nodes to estimate occult tumor burden. Indeed, recursive partitioning based on GUCY2C qRT-PCR and time to recurrence stratified this patient population into specific risk categories in which 45% of pN0 patients exhibited low (3%), 40% intermediate (>50%), and 15% high (˜80%) risk of disease recurrence (p<0.001; FIG. 2). A similar analysis based on disease-free survival also stratified this population into specific risk categories in which 39% of pN0 patients exhibited low (3%), 27% intermediate (22%), and 34% high (66%) risk of disease recurrence (p<0.001). This is a striking enhancement of the use of GUCY2C as a categorical (yes/no) marker, where only 12% of patients were negative, with a low (6%) risk, while 88% of patients were positive with an intermediate (20%) risk. These observations highlight the diagnostic opportunity to quantify occult tumor burden by GUCY2C qRT-PCR and recursive partitioning to assign risk in patients with pN0 colorectal cancer. In that context, identification of cohorts with a risk equivalent to patients with stage IV colorectal cancer, who have distant metastases, underscores the predictive value of this molecular staging algorithm. Indeed, it is tempting to speculate that patients with the greatest tumor burden and a risk of recurrence of >80% might benefit from adjuvant therapy. Here, GUCY2C qRT-PCR and recursive partitioning will be employed to assess the distribution of occult tumor burden, associated with excess risk, in pN0 African Americans and Caucasians.

### Example 2

The present analysis defines the association between occult tumor burden in lymph nodes, estimated by GUCY2C qRT-PCR, and time to recurrence and disease-free survival in patients with pN0 colorectal cancer.

Lymph node involvement by histopathology informs colorectal cancer prognosis, whereas recurrence in 25% of node-negative patients suggests the presence of occult metastasis. GUCY2C (guanylyl cyclase C) is a marker of colorectal cancer cells that identifies occult nodal metastases associated with recurrence risk. Here, the association of occult tumor burden, quantified by GUCY2C reverse transcriptase-PCR (RT-PCR), with outcomes in colorectal cancer is defined.

Lymph nodes (range: 2-159) from 291 prospectively enrolled node-negative colorectal cancer patients were analyzed by histopathology and GUCY2C quantitative RT-PCR. Participants were followed for a median of 24 months (range: 2-63). Time to recurrence and disease-free survival served as primary and secondary outcomes, respectively. Association of outcomes with prognostic markers, including molecular tumor burden, was estimated by recursive partitioning and Cox models.

In this cohort, 176 (60%) patients exhibited low tumor burden (MolLow), and all but four remained free of disease [recurrence rate 2.3% (95% CI, 0.1-4.5%)]. Also, 90 (31%) patients exhibited intermediate tumor burden (Mol_{Int}) and 30 [33.3% (23.7-44.1)] developed recurrent disease. Furthermore, 25 (9%) patients exhibited high tumor burden (Mol_{High}) and 17 [68.0% (46.5-85.1)] developed recurrent disease (P<0.001). Occult tumor burden was an independent marker of prognosis. Mol_{Int} and Mol_{High} patients exhibited a graded risk of earlier time to recurrence [Mol_{Int}, adjusted HR 25.52 (11.08-143.18); P<0.001; Mol_{High}, 65.38 (39.01-676.94); P<0.001] and reduced disease-free survival [Mol_{Int}, 9.77 (6.26-87.26); P<0.001; Mol_{High}, 22.97 (21.59-316.16); P<0.001].

Molecular tumor burden in lymph nodes is independently associated with time to recurrence and disease-free survival in patients with node-negative colorectal cancer.

### Methods

### Study Design

This prospective observational trial at 9 centers in the United States and Canada explored the prognostic utility of GUCY2C qRT-PCR in lymph nodes of pN0 colorectal cancer patients. Investigators and clinical personnel were blinded to results of molecular analyses, whereas laboratory personnel and analysts were blinded to patient and clinical information. To have at least 80% power to detect a HR of 1.6 (P≦0.05, 2-sided) employing categorical assessment of occult tumor metastases, 225 pN0 patients were required. The study protocol was approved by the Institutional Review Board of each participating hospital. The 291 pN0 patients who met eligibility criteria provided 7,310 lymph nodes (range: 2-159, median 21 lymph nodes per patient) for histopathologic examination, of which 2,774 nodes (range: 1-87, median 8 lymph nodes per patient) were obtained by fresh dissection and eligible for analysis by qRT-PCR. Disease status, obtained in routine follow-up by treating physicians, was provided for all patients through Dec. 31, 2009.

### Patients and Tissues

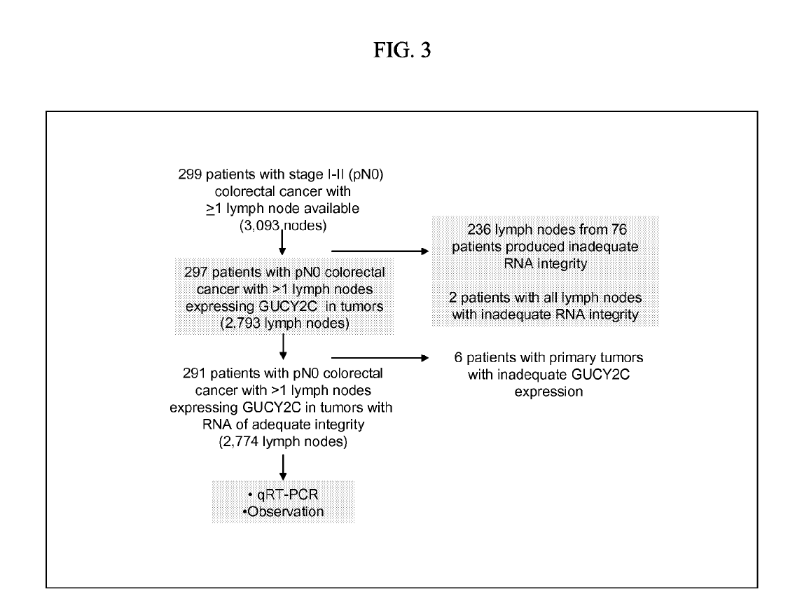

Between March 2002 and June 2007, 299 stages 0 to II pN0 colorectal cancer patients who provided informed consent in writing prior to surgery at one of 7 academic medical centers and 2 community hospitals in the United States and Canada (FIG. 3) were enrolled. Patients were ineligible if they had a previous history of cancer, metachronous extraintestinal cancer, or perioperative mortality associated with primary resection. For all eligible patients, preoperative and perioperative examinations revealed no evidence of metastatic disease. Lymph nodes and, when available, tumor specimens (51%) were dissected from colon and rectum resections and frozen at −80° C. within 1 hour to minimize warm ischemia. Half of each resected lymph node was fixed with formalin and embedded in paraffin for histopathologic examination. Lymph node specimens were subjected to molecular analysis if (i) tumor samples, where available, expressed GUCY2C mRNA above background levels in disease-free lymph nodes (>30 copies) and (ii) at least 1 lymph node was provided which yielded RNA of sufficient integrity for analysis. Thus, analysis of the 3,093 lymph nodes available from the 299 pN0 patients revealed 236 nodes from 76 patients yielding RNA of insufficient integrity by (β-actin qRT-PCR, excluding 2 patients (FIG. 3). Moreover, GUCY2C expression in tumors was below background levels in 6 patients who were excluded from further analysis.

### RNA Isolation

RNA was extracted from tissues by a modification of the acid guanidinium thiocyanate-phenol-chloroform extraction method. Briefly, individual tissues were pulverized in 1.0 mL TRI Reagent (Molecular Research Center) with 12 to 14 sterile 2.5 mm zirconium beads in a bead mill (Biospec) for 1 to 2 minutes. Phase separation was done with 0.1 mL bichloropropane, and the aqueous phase reextracted with 0.5 mL chloroform. RNA was precipitated with 50% isopropanol and washed with 70% ethanol. Air-dried RNA was dissolved in water, concentration determined by spectrophotometry, and stored at −80° C.

### RT-PCR

GUCY2C mRNA was quantified by RT-PCR employing an established analytically validated assay. The EZ RT-PCR kit (Applied Biosystems) was employed to amplify GUCY2C mRNA from total RNA in a 50 μL reaction. Optical strip tubes were used for all reactions, which were conducted in an ABI 7000 Sequence Detection System (Applied Biosystems). In addition to the kit components [50 mmol/L Bicine (pH 8.2), 115 mmol/L KOAc, 10 μmol/L EDTA, 60 nmol/L ROX, 8% glycerol, 3 mmol/L Mg (OAc)2, 300 μmol/L each dATP, dCTP, and dGTP, 600 μmol/L dUTP, 0.5 U uracil N-glycosylase, and 5 U rTth DNA polymerase], the reaction master mix contained 900 nmol/L each of forward (SEQ ID NO:1 ATTCTAGTGGATCTTTTCAATGACCA) and reverse primers (SEQ ID NO:2 CGTCAGAACAAGGACATTTTTCAT), 200 nmol/L TaqMan probe (FAM-TACTTGGAGG-ACAATGTCACAGCCCCTG-TAMRA), and 1 μg RNA template. The housekeeping gene β-actin was amplified employing similar conditions except that forward (SEQ ID NO:3 CCACACTGTGCCCATCTACG) and reverse (SEQ ID NO:4 AGGATCTTCATGAGGTAGTCAGTCAG) primers were 300 nmol/L each, whereas the TaqMan probe [FAMATGCCC-X(TAMRA)-CCCCCATGCCATCCTGCGTp] was 200 nmol/L. The thermocycler program employed for reverse transcription included: 50 degree×2 minutes, 60 degree×30 minutes, 95 degree×5 minutes, and for PCR: 45 cycles of 94 degree×20 seconds, 62 degree×1 minute. Reactions were conducted at least in duplicate and results averaged.

### Statistical Methods

Statistical methods for estimating GUCY2C and β-actin mRNA by logistic regression analysis is described below. The primary clinical endpoint was time to recurrence, measured from the date of surgery to the time of the last follow-up, recurrence event, or death. Disease-free survival, defined as time from surgery to any event regardless of cause, was a secondary outcome. Date of recurrence was established by radiographic studies, laboratory studies, physical examination, and/or histopathology. CIs for raw survival rates were computed by the exact method of Clopper-Pearson.

Recursive partitioning, a tree-branching algorithm that identifies homogeneous cohorts in populations, served as the primary analytic approach for survival outcomes, implemented in the R routine RPART. This algorithm tests, across all possible variables and levels, for the variable which optimally identifies discrete groups within the study population. The process repeats recursively until a stopping criterion, predefined here as the software default of any subgroup with fewer than 20 participants, is achieved. Cross-validation (10-fold) during model fitting provided model stability and accuracy and avoided over-fitting. This algorithm was applied using quantitative measures of occult tumor burden as variables for risk stratification. Metrics of occult tumor burden by GUCY2C qRT-PCR included median copy number, maximum copy number, median relative (normalized to β-actin) expression, maximum relative expression, total copy number, and total relative expression across lymph nodes, and the total number of GUCY2C-positive lymph nodes quantified. Time to recurrence or disease-free survival served as outcomes in these analyses. Categories of low, medium, and high risk for time to recurrence and disease-free survival were defined by amalgamation.

Survival distributions for patients in different risk strata were compared employing the log-rank test. Although Kaplan-Meier plots display censored survival at 36 months, analyses incorporated all events up to the date of last follow-up. Simultaneous prognostic effects of risk categories and additional covariates were estimated employing Cox regression analysis. Established prognostic variables in the Cox model for recurrence included T stage, grade, lymphovascular invasion, receipt of chemotherapy and/or radiotherapy, anatomic location, number of lymph nodes harvested for histopathology (≦12, <12), and tumor burden risk status defined from recursive partitioning analysis. The multivariable model for each outcome included all of the recognized prognostic measures regardless of significance to establish the additional independent prognostic effect of occult tumor burden. Because selection of optimal cut-points and subsequent Cox modeling is known to yield inflated alpha level testing, 5,000 bootstrap samples were utilized to establish adjusted CIs and empirical P values. Although comparable as internal validation techniques, bootstrapping is preferred here to cross-validation reflecting limitations in populations and events due to cohort segmentation inherent in the latter approach. The sensitivity of Cox models employing categorical (yes/no) analysis of occult metastases versus occult tumor burden (how much) were compared by using the Akaike Information Criteria (AIC). A global test of proportional hazards for each of the Cox models was completed according to Hosmer and Lemeshow. All tests were 2-sided and P<0.05 was considered statistically significant. All analyses were done with R v 2.9.2, SAS v9.2, and Stata v11.0.

### Results

### Patient Characteristics

The 291 pN0 patients had a mean age of 68 years (26-90 years) at diagnosis and 55% were male (Table 1). Clinicopathologic features, including depth of tumor penetration (T1/2, T3, and T4), and tumor anatomic location (right, left, and sigmoid colon) were similar to national experience. Patients with colon cancer represented 85.9%, whereas those with rectal tumors comprised 14.1%.

### Occult Tumor Burden and Disease Recurrence

Clinical outcomes in pN0 colorectal cancer patients were analyzed by recursive partitioning by using metrics of occult tumor burden estimated by GUCY2C qRT-PCR. The median of relative GUCY2C expression across patient nodes was the dominant quantitative variable stratifying risk. Partitioning algorithms also utilized the maximum relative expression across nodes, the number of positive nodes, the median absolute GUCY2C copy number, and the total absolute copy number across nodes to establish risk categories.

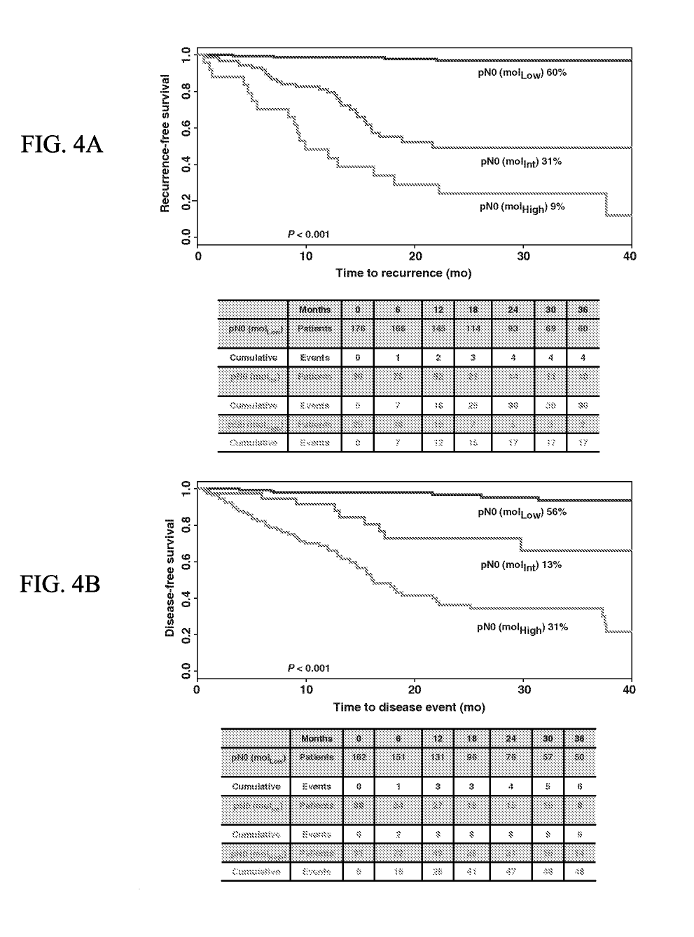

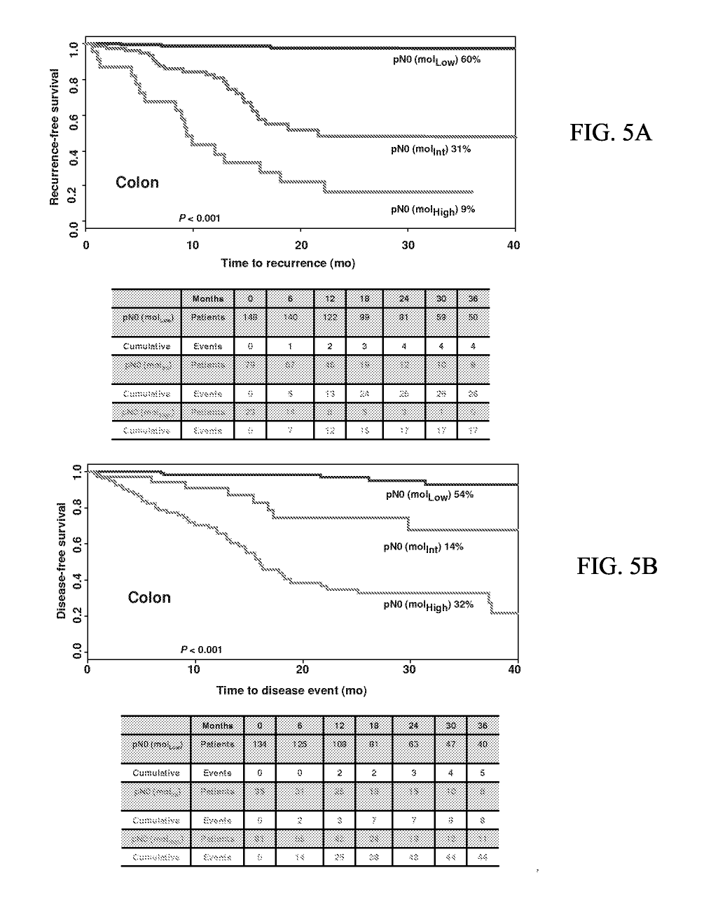

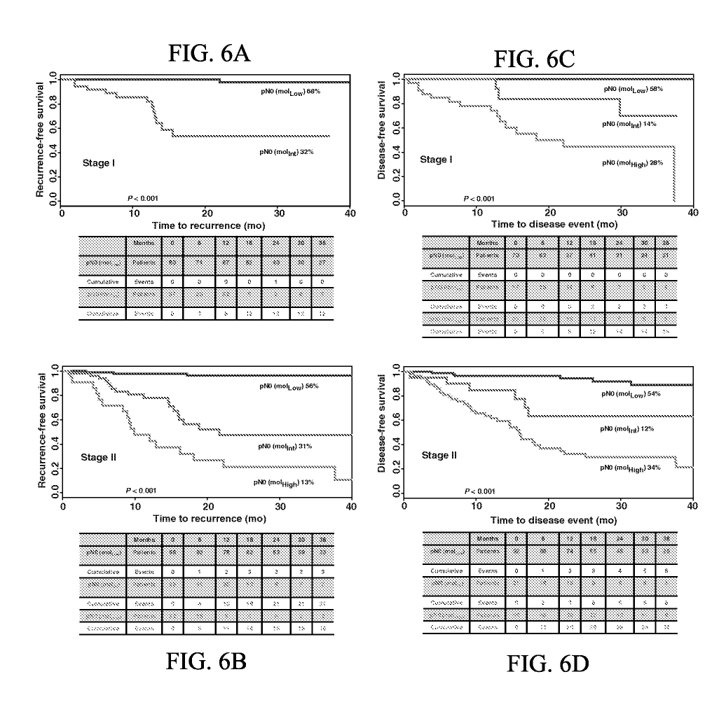

On the basis of time to recurrence, GUCY2C qRT-PCR stratified pN0 patients into categories in which 176 (60%) patients exhibited low (MolLow), 90 (31%) exhibited intermediate (MolInt), and 25 (9%) exhibited high (MolHigh; P<0.001) risk of disease recurrence (FIG. 4). Median follow-up was 25 months (range: 2-62) for MolLow, 19 months (range: 1-61) for MolInt, and 25 months (range: 1-63) for MolHigh patients. All but 4 of the MolLow patients remained free of disease during follow-up [recurrence rate 2.3% (95% CI, 0.1-4.5)]; 30 [33.0% (23.7-44.1)] MolInt patients developed recurrent disease; and 17 [68.0% (46.5-85.1)] MolHigh patients developed recurrent disease (P<0.001; FIG. 4). Subgroup analyses revealed that occult tumor burden conferred a substantially worse time to recurrence among patients with colon cancer (FIG. 5), AJCC stages I and II disease (FIG. 6), 3 or more years of follow-up, or optimal collections 12) of lymph nodes.

Similarly, based on disease-free survival, GUCY2C qRT-PCR stratified this population in which 162 (56%) were MolLow, 38 (13%) MolInt, and 91 (31%) MolHigh (P<0.001; FIG. 4). For disease-free survival, median follow-up was 24 months (range: 2-62) for MolLow, 25 months (range: 1-59) for MolInt, and 24 months (range: 1-63) for MolHigh patients. All but 6 of the MolLow patients remained free of disease during follow-up [3.7% (0.8-6.6)]; 9 [23.7% (6.9-10.2)] MolInt patients developed disease-related events; and 48 [52.8% (42.5-63.0)] MolHigh patients developed disease-related events (P<0.001; FIG. 4). Like time to recurrence, subgroup analyses suggest that occult tumor burden predicted reduced disease-free survival in patients with colon cancer (FIG. 5), or disease with different stages (FIG. 6), duration, or lymph node collections.

### Occult Tumor Burden as a Prognostic Variable

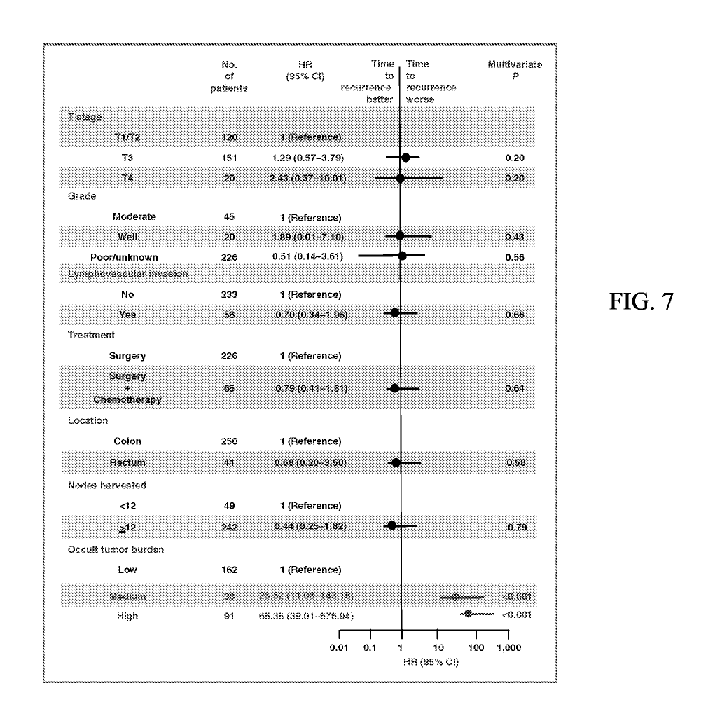

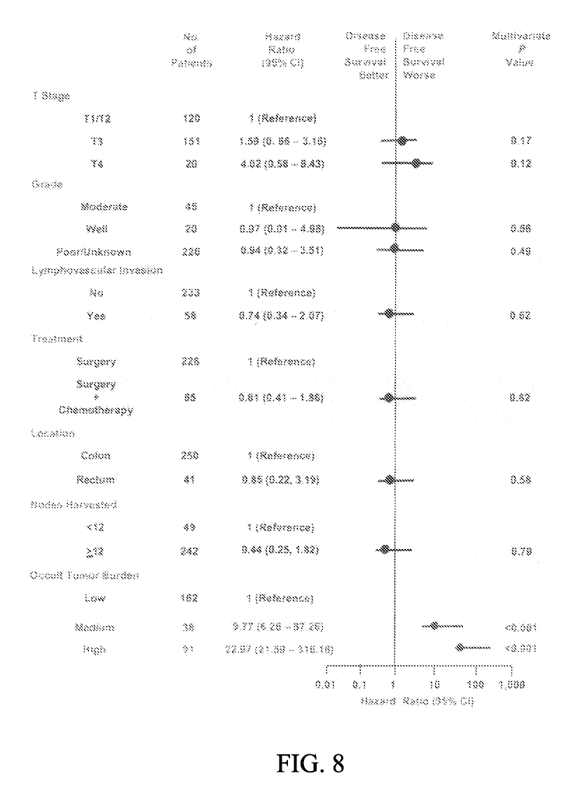

Multivariable analyses employing Cox proportional hazards models (FIG. 7 and FIG. 8) revealed that canonical prognostic clinicopathologic features contributed little as independent markers of recurrence risk in patients with pN0 colorectal cancer. However, occult tumor burden in lymph nodes provided independent prognostic information. The global test of nonproportional hazards for time to recurrence (X², 6.93; 10 df; P=0.73) and disease-free survival [X²,10.99; 10 df; P=0.36) indicated that there were no significant departures from the proportional hazards assumptions of these models. Patients who were Mol_{Int} exhibited time to recurrence [adjusted HR 25.52 (11.08-143.18); P=0.001; FIG. 7] and disease-free survival [adjusted HR 9.77 (6.26-87.26); P=0.001; FIG. 8) comparable with published results for stage III patients. Patients who were Mol_{High} exhibited time to recurrence [adjusted HR 65.38 (39.01-676.94); P<0.001; FIG. 7] and disease-free survival [adjusted HR 22.97 (21.59-316.16); P<0.001; FIG. 8] that approach survival characteristics for patients with stage IV colon cancer. Sensitivity analysis revealed that Cox models employing risk categories for time to recurrence established by occult tumor burden were substantially superior (AIC, 470.2) to those employing categorical (yes/no) analysis of occult metastases (AIC, 561.9). Similarly, Cox models employing risk categories for disease-free survival established by occult tumor burden were considerably preferred (AIC, 625.6) over those employing categorical analysis of occult metastases (AIC, 699.7).

### Discussion

A widely held tenet of cancer staging is the relationship between regional lymph node metastases and prognostic risk. In colorectal cancer, lymph node metastasis is the single most important prognostic characteristic, representing pathologic evidence of tumor dissemination beyond its primary location. Clinically, approximately 50% of stage III patients will suffer disease recurrence. Because up to 25% of patients with lymph nodes free of tumor involvement also suffer recurrent disease, it is presumed that many such patients harbor occult metastases not identified at the time of primary resection.

Understaging by conventional methods reflects sampling inadequacies inherent in analyzing small volumes of tissue from insufficient lymph node collections, and the insensitivity of histopathology, which reliably detects only 1 cancer cell in 200 normal cells. Molecular staging can overcome these limitations in the detection of occult lymph node metastases by incorporating all available tissue into analyses and increasing detection sensitivity through quantifiable, highly sensitive, and disease-specific molecular markers.

Prospective, categorical (yes/no) detection of GUCY2C expression in regional lymph nodes was shown to be an independent prognostic marker of recurrence risk in pN0 colorectal cancer patients. The current results highlight the dramatic enhancement in diagnostic specificity achieved by quantifying molecular tumor burden. When employed as a categorical marker, only 13% of GUCY2C-negative patients were free of occult metastases, but their recurrence risk was low (6%). Although recurrence risk was significantly higher (20%) in the 87% of patients who were GUCY2C positive, most of them did not suffer recurrence. It is apparent that nodal metastases, detected by any method, do not assure recurrence; rather, they indicate risk. For example, not all stage III patients who by definition have detectable lymph node metastases, ultimately develop recurrent disease.

Beyond the categorical presence of metastases, there is an evolving relationship between the quantity of tumor cells in lymph nodes and prognostic risk of recurrence. There is already a well-established correlation between burden of disease, quantified as the number of lymph nodes harboring tumor cells by histopathology and prognostic risk in colorectal cancer patients. Assuming there are adequate numbers of nodes to review, stage III patients with 4 or more involved lymph nodes exhibit a recurrence rate that is approximately 50% to 100% greater than those with 3 or less involved nodes.

In addition to the number of involved lymph nodes, there is an association between the volume of cancer cells in individual nodes, disease burden, and prognostic risk. Although metastatic foci of 0.2 mm or greater are associated with increased disease recurrence, the relationship between individual tumor cells or nests smaller than 0.2 mm and prognostic risk remains undefined. The emergence of qRT-PCR provides an unprecedented opportunity for cancer cell enumeration, offering a molecular analogue of the morphologic assessment of metastatic volumes by histopathology. Furthermore, quantifying occult metastases in a large volume of tissue (the entire sample), rather than a thin section, and mapping those metastases across the lymph node network enhances 2-dimensional morphology, providing estimates of molecular tumor burden.

The results suggest that the patients with greater occult tumor burden in lymph nodes, estimated by GUCY2C qRT-PCR, have a greater risk of recurrence compared with patients with less tumor burden. In the setting of a common malignancy such as colorectal cancer, the quantification of occult tumor burden in lymph nodes to estimate prognostic risk has not been explored previously. Furthermore, the relevant qRT-PCR parameters to estimate tumor burden have not been defined. In the absence of prior experience, recursive partitioning was employed to objectively identify, without bias, parameters that define subgroups of prognostic risk in pN0 patients. Recursive partitioning, applied to all patients using measures of tumor burden established by GUCY2C qRT-PCR stratified pN0 patients into a low-risk cohort representing approximately 50% to 60% of the population, with a very low (<5%) incidence of disease recurrence, an intermediate-risk cohort with an incidence of disease recurrence of approximately 33%, and a high-risk cohort with more than 60% incidence of recurrence. Multivariable analyses revealed that molecular tumor burden was a powerful independent prognostic marker of time to recurrence and disease-free survival in the context of well-established prognostic clinicopathologic characteristics.

Colon and rectal cancers were considered together in this analysis because GUCY2C is a molecular marker for metastatic tumor cells of intestinal origin and identifies occult tumor burden in patients with either of these diseases. Colon cancers were analyzed as a separate cohort, whereas rectal cancer patients were a small minority of the total, providing insufficient numbers for recursive partitioning and risk group analysis. It is noteworthy that the treatment of some rectal cancer patients with neoadjuvant chemoradiotherapy would bias the analysis against the working hypothesis. Indeed, this treatment could produce false negative results, reflecting the absence of adequate lymph node collections for analysis or eradication of occult tumor cells in lymph nodes. However, even in the context of this potential negative bias, the analysis of the full cohort revealed a strong correlation between occult tumor burden and prognostic risk. These results argue for a separate analysis of an adequate population of rectal cancer patients to confirm the utility of occult tumor burden to stratify prognostic risk in these patients.

Tumor burden assessed by GUCY2C qRT-PCR compares favorably with recent gene expression-based efforts to predict colorectal cancer recurrence. Quantification of expression of a 12-gene panel in tumors (Oncotype DX Colon-Cancer; Genomics Health) stratified 711 stage II (pN0) colon cancer patients into categories in which 40% of patients exhibited a minimum 12% risk, 26% had a maximum 22% risk, whereas 34% had a risk intermediate between that minimum and maximum, at 36 months. Superior specificity, where approximately 60% of pN0 patients exhibit near-zero risk of recurrence, coupled with a greater demonstrable range of recurrence risk, in the context of a single molecular marker, suggests that quantifying molecular tumor burden by GUCY2C qRT-PCR may offer a diagnostic approach with performance characteristics not previously achieved.

The presence of tumor cells in regional lymph nodes also directs therapy in patients with colon cancer. Although adjuvant chemotherapy provides a survival benefit to patients with stage III disease, its utility in patients with pN0 colon cancer remains uncertain, with marginal survival benefits in stage II patients in some, but not all, clinical trials. This uncertainty of treatment benefit is shown in the evolution of treatment guidelines, in which adjuvant therapy has become discretionary in stage II patients with clinicopathologic features of poor prognostic risk, including T4 stage, intestinal obstruction, and intestinal perforation. Heterogeneous responses to therapy in pN0 patients may reflect, in part, the variable presence of occult metastases. Moreover, standard of care includes adjuvant chemotherapy for stage III patients. It is tempting to speculate that MolInt and MolHigh patients, with survival characteristics approximating stage III and IV colon cancers, respectively, might derive benefit from adjuvant therapy. These considerations highlight the importance of advancing beyond the present study to refine the predictive utility of quantifying molecular tumor burden by GUCY2C qRT-PCR. Molecular assessments such as GUCY2C analysis could better inform the use of adjuvant chemotherapy in pN0 patients.

In summary, GUCYC2C qRT-PCR analysis of resected lymph nodes in pN0 colorectal cancer patients revealed 3 discrete strata of recurrence risk ranging from less than 5% to greater than 60%. These results show, for the first time, the impact of quantitative occult tumor burden estimates on clinical prognosis. They underscore the importance of continuing to validate this novel approach by establishing threshold tumor burden values that can be broadly applied to risk estimation in colorectal cancer patients. Also, they highlight the significance of quantifying the number of lymph nodes required for optimal molecular tumor burden assessment. This molecular approach to occult tumor burden assessment provides a unique opportunity to define the constellation of tumor (microsatellite instability, mutations, methylation, and chromosomal instability) and lymph node parameters that optimally estimate prognostic risk of individual patients. Moreover, it establishes the importance of defining the contribution of these molecular approaches to therapeutic decision making for node-negative colorectal cancer patients.

### Supplemental Information

### Relative Quantification of GCC Expression by QRT-PCR

GCC and β-actin expression was estimated by logistic regression analysis of amplification profiles from individual RT-PCR reactions, providing an efficiency-adjusted relative quantification based on parameter estimates from the fitted models which reduces bias and error.¹⁹ In the re-parameterized logistic model:

$$F ( x ) = L + \frac{U - L}{1 + {e}^{m} {A}^{- x}} , \qquad ( 1 )$$

where L and U=L+PK are lower and upper asymptotes, respectively, A is the maximum amplification rate, and m=ln(K/N(0)−1), where N(0) is the number of starting templates in the reaction, m may be used to compute the log-ratio expression of a target gene normalized to a reference gene. For real RT-PCR reactions, N(0) is less than K by orders of magnitude, and therefore

m=ln(K/N(0)−1)≈ln(K)−ln(N(0)),

where K may either be the same for target and reference reactions, or, at least, the same constant for all target reactions and another constant for all reference reactions. Hence, up to a constant shift, common for all reactions, the log-ratio of a target normalized to a reference may be computed as

ln R_{T/R}=ln N_{T}(0)−ln N_{R}(0)>>m_{R}−m_{T} (2)

where m_{T} and m_{R} are m parameters in model (1) for target and reference gene reactions, respectively.

If one considers the nonlinear model for fluorescence Fᵢ at cycle xᵢ:

$${F}_{i} = L + \frac{U - L}{1 + {e}^{m} {A}^{- {x}_{i}}} + {ɛ}_{i} , \qquad ( 3 )$$

where εᵢ˜i.i.d. N(0,σ) represent measurement errors. Fitting (3) using standard non-linear regression methods provides the estimates \hat{m}_{T} and \hat{m}_{R} and their standard errors, se(\hat{m}_{T}) and se(\hat{m}_{R}) for each target and reference gene reaction. Then the log-ratio of a target normalized to a reference is estimated as:

Ĩñ\tilde{R}\tilde{InR_{T/R}}=\hat{m}_{R}−\hat{m}_{T} (4)

and the standard error of Ĩñ\tilde{R}\tilde{InR_{T/R}} is computed as

se[Ĩñ\tilde{R}\tilde{InR_{T/R}}]=\sqrt{[se(\hat{m}_{T})]²+[se(\hat{m}_{R})]²}. (5)

Here, the qRT-PCR fluorescence profile for GCC and beta-actin for each lymph node was exported to Excel data files, imported to SAS, and fit using model (3) with the Nonlin procedure. Parameter estimates, measures of goodness of fit and convergence status were recorded for each reaction and used for further analysis. Each lymph node was run for each gene in duplicate, and averages for each node computed. In that context, for n_{T} replicates of target and n_{R} replicates of reference RT-PCR reactions for the same biological sample, let \hat{m}_{Ti} i=1, . . . , n_{T} and \hat{m}_{Ri}, i=1, . . . , n_{R} be non-linear regression estimates of parameter m from model (3) with the corresponding estimated standard errors se(\hat{m}_{Ti}) i=1, . . . , n_{T} and se(\hat{m}_{Ri}) i=1, . . . , n_{R}.

Denote

$${\overline{m}}_{T} = \frac{1}{{n}_{T}} {\sum }_{i = 1}^{{n}_{T}} {\hat{m}}_{Ti}$$

$${\overline{m}}_{R} = \frac{1}{{n}_{R}} {\sum }_{i = 1}^{{n}_{T}} {\hat{m}}_{Ri} .$$

For the same biological sample, replicates are considered independent, conditional on the random effect of a sample or an individual. The log-ratio and its standard error may be computed as:

$$\hat{ln {R}_{T / R}} = {\overline{m}}_{R} - {\overline{m}}_{T} \\ se [ \hat{ln {R}_{T / R}} ] = \sqrt{\frac{1}{{n}_{T}^{2}} {\sum }_{i = 1}^{{n}_{T}} {[ se ( {\hat{m}}_{Ti} ) ]}^{2} + \frac{1}{{n}_{R}^{2}} {\sum }_{i = 1}^{{n}_{R}} {[ se ( {\hat{m}}_{Ri} ) ]}^{2}} . \qquad ( 6 )$$

Here, relative GCC expression was computed for each lymph node for each patient using this approach. For any reaction where the logistic model did not converge, or did not exhibit goodness of fit measuring ≧80%, or if the amplification constant, A in model (1), was not ≧1.5, the fluorescence isotherms were individually reviewed by two members of the research team. In all cases where this occurred for GCC, reactions did not amplify, implying zero or low expression of the gene. For the same lymph node, if β-actin expression was >2000 copies, representing the 5^{th} percentile of beta-actin expression¹⁴, then it was presumed the sample had viable RNA, and GCC expression was set to the lowest measured value of GCC expression. Nodes where β-actin expression <2000 copies were eliminated from further analysis.

The distribution of relative GCC expression for each lymph node was quantified, averaged over replicates, and the median computed. As a conservative approach for this analysis, nodes where relative GCC expression was ≧median were considered positive, while those <median were considered negative. Median expression was specifically selected a priori as the threshold because it maximizes the probability of identifying patients harboring occult metastases in context of variable collections of lymph nodes from individual patients. In this analysis, median expression was estimated as about 173 copies of GCC mRNA, closely approximating that obtained in earlier studies (about 200 copies) employing different samples and analytic approaches, reinforcing the validity of the techniques. Employing this threshold provides a sensitivity and specificity of 93% and 78%, respectively, when applied to the validation cohort of true positive and negative lymph nodes defined previously. Lymph nodes for each patient were then summarized to compute the number of positive lymph nodes. For Kaplan-Meier and Cox analyses, this was categorized as zero nodes positive=pN0[mol−] or ≧1 nodes positive=pN0[mol+]. In an additional subgroup where >12 lymph nodes were available for each patient, the categories 0 to 3 lymph nodes positive and ≧4 lymph nodes positive were applied, which are comparable to those employed in histopathological staging and risk stratification in colorectal cancer.^{3,23}

### Example 3

There is an ever-widening racial gap in mortality from colorectal cancer, the 4th most common incident cancer and the 2nd leading cause of cancer death in the U.S. For example, while disease-specific mortality has decreased 54% for non-Hispanic white (white) men, non-Hispanic black (black) men have experienced an increase of 28%, since 1960. Racial differences in mortality reflect tumor clinicopathologic characteristics, including advanced stage of disease at diagnosis associated with poorer outcomes in black, compared to white, patients. In turn, differences in disease stage at diagnosis reflect disparities in socioeconomic status and access to quality health service. However, tumor characteristics, socioeconomic status and health services access contribute only about 50% to excess mortality reflecting race. Other factors underlying race-based excess mortality in colorectal cancer remain undefined.

Beyond clinicopathological differences at diagnosis, there is an under-appreciated racial disparity in stage-specific mortality in colorectal cancer. For patients with regionally-advanced disease (lymph node-positive; Stage III), blacks experience 10% excess mortality compared to whites. This difference is further amplified in patients with local disease (lymph node-negative (pN0); Stage I and II) where blacks exhibit 40% excess mortality compared to whites. Unlike overall disease mortality, socioeconomic status contributes negligibly to racial disparities in stage-specific outcomes. Indeed, beyond the 50% contribution of traditional clinicopathologic characteristics, socioeconomic status, and health services access, stage-specific disparities may be one primary driver of overall differences in mortality in blacks and whites with colorectal cancer. In turn, the precise factors contributing to racial differences in stage-specific mortality have not been defined. However, the predominance of this racial gap in the earliest stages (pN0) of disease, which receive minimal post-surgical intervention suggests contributions by factors other than therapeutic application, acceptance, or compliance.

The quantity of occult tumor burden across the regional lymph node network stratifies risk, identifying patients with near-zero risk, those with elevated risk of 33%, and those with 70% risk, of unfavorable outcomes. The association of disparities in outcomes in black and white patients with pN0 colorectal cancer distinguished by differences in occult tumor burden in regional lymph nodes, estimated by GUCY2C RT-qPCR is defined here.

Data from the study described in Example 2 was used in a subsequent analysis to explore the association of racial differences in outcomes in pN0 patients with occult tumor burden in lymph nodes. The data in Example 2 refers to lymph nodes from 291 patients. In the subsequent analysis exploring racial differences disclosed here, data from nine patients were excluded.

Thus, in the analysis exploring racial differences in outcomes in pN0 patients with occult tumor burden in lymph nodes, lymph nodes (range: 2-159) from 282 prospectively enrolled pN0 colorectal cancer patients were analyzed by GUCY2C quantitative RT-(q)PCR and followed for a median of 24 months (range: 2-63). Risk categories defined using occult tumor burden was the primary outcome measure. Association of prognostic variables and risk were defined by multivariate polytomous logistic regression. Occult tumor burden stratified this cohort of 259 white and 23 black patients into categories with low (60%; recurrence rate (RR)=2.3% [95% CI 0.1-4.5%]), intermediate (31%; RR=33.3% [23.7%-44.1%]), and high (9%; RR=68.0% [46.5%-85.1%], p<0.001) risk. Black, compared to white, patients exhibited 4-fold greater occult metastases in individual nodes (p<0.001). Multivariable analysis revealed that race (p=0.02), T stage (p=0.02), and number of nodes collected (p=0.003) were independent prognostic markers. Black, compared to white, patients were more likely to harbor levels of occult tumor burden, associated with the highest recurrence risk (adjusted odds ratio=6.00 [1.69-21.39]; p=0.006). Thus, racial disparities in stage-specific outcomes in colorectal cancer are associated with differences in occult tumor burden in regional lymph nodes. Refining the prognostic utility of occult tumor burden can guide therapeutic decision making that eliminates the racial gap in stage-specific mortality in colorectal cancer.

### Methods

The basic study design, patients and tissues, RNA isolation, and RT-PCR are disclosed in Example 2. As noted above, the initial analysis of the lymph nodes available from the 299 criteria eligible pN0 patients resulted in eight patients being excluded from the study due to RNA of insufficient integrity by β-actin (two patients) and GUCY2C expression in tumors was below background levels (six patients). The analysis in Example 2 is thus based upon data from 291 patients. Data from nine additional patients was excluded in this subsequent analysis directed at impact of the number of lymph nodes analyzed to the accuracy of the risk stratification; the nine patients excluded were not identified as white or black. The analyses were performed on data from the 282 patients identified as white or black.

A linear mixed effects model of expression across all nodes from all eligible patients included random effect of patient, and fixed effects of center, and race. The primary clinical endpoint was molecular risk category (low, intermediate, high), based on time to recurrence and recursive partitioning analysis. Confidence intervals for raw survival rates were computed by the exact method of Clopper-Pearson. All tests were two-sided, and p<0.05 was considered statistically significant. All analyses were performed with R v 2.11.2, SAS v9.2.

Univariable analysis of association of molecular risk category with demographic and prognostic factors was completed using the chi-square test of association. Multivariable analyses using polytomous logistic regression employed risk level and established prognostic variables including T stage, grade, lymphovascular invasion, receipt of chemotherapy and/or radiotherapy, anatomical location, number of lymph nodes collected for histopathology, and race. Initial multivariable models included all established prognostic measures regardless of significance and a manual backwards stepwise approach was used to establish the final model of association with occult tumor burden risk level. Variables with the least association with outcome were removed one at a time until all remaining variables were significant by a Type 3 test of association at p<0.05. Predicted conditional probabilities and 95% two-sided confidence intervals were estimated from the final multivariable model. These probabilities are reported to demonstrate the contribution of each variable to the final model of molecular risk strata. Exact adjusted odds ratios were calculated and reported for factors with small cell sizes in multivariable models, when appropriate.

### Results

### Patient Characteristics.

Of the 282 pN0 patients, black patients comprised 7.9% of the total population enrolled, nearly identical to the national average for disease-specific racial distribution. There were no significant differences in clinicopathologic characteristics between black and white patients (Table 2).

### Occult Tumor Burden and Risk Stratification.

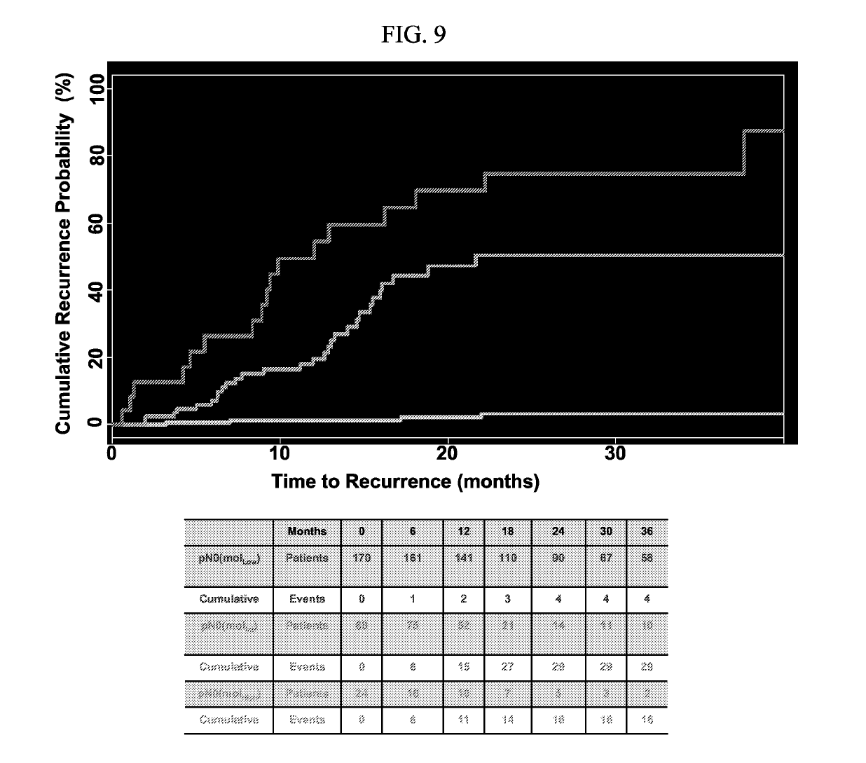

Clinical outcomes in pN0 colorectal cancer patients were analyzed by recursive partitioning using metrics of occult tumor burden estimated by GUCY2C RT-qPCR. Based on time to recurrence, GUCY2C RT-qPCR stratified pN0 patients into categories in which 170 (60%) patients exhibited low (MolLow), 88 (31%) exhibited intermediate (MolInt), and 24 (9%) exhibited high (MolHigh) (p<0.001) risk of disease recurrence (FIG. 9). All but 4 of the MolLow patients remained free of disease during follow-up (recurrence rate (RR)=2.3% [95% CI 0.1-4.5%]); 29 MolInt patients developed recurrent disease (RR=33.3% [23.7%-44.1%]); and 16 RR=68.0% [46.5%-85.1%]) MolHigh patients developed recurrent disease (p<0.001; FIG. 9). Univariate analysis revealed the expected relationship between advanced T stage, occult tumor burden and risk (p=0.008). Similarly, the accuracy of molecular staging was improved by collecting 13 or more lymph nodes from each patient (p=0.002) recapitulating established enhancements in histopathologic staging by increased nodal harvests.

### Occult Tumor Burden and Race.

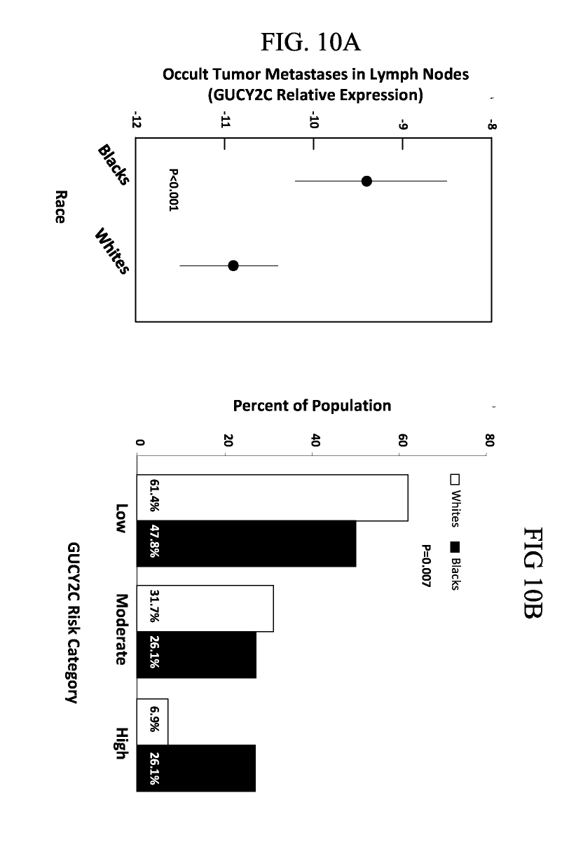

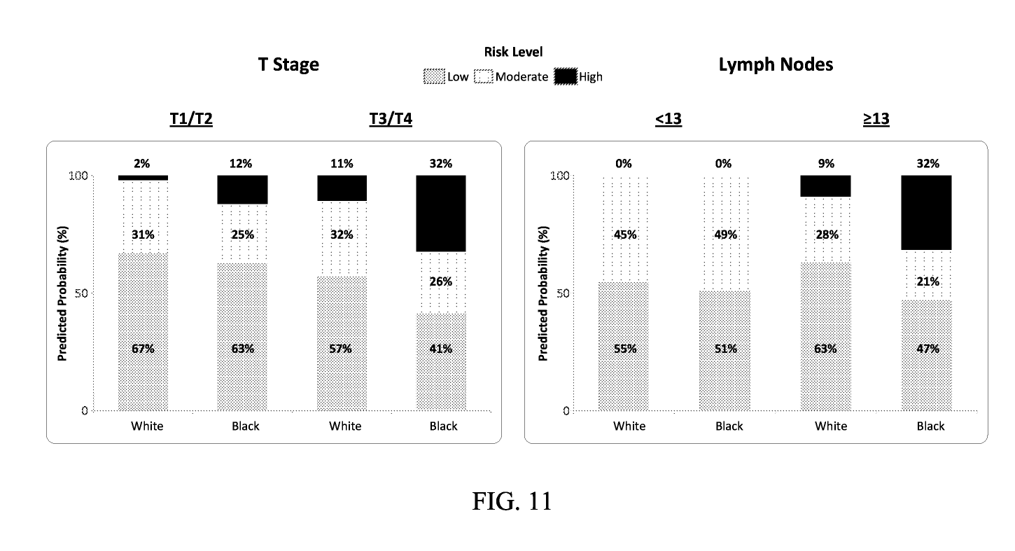

Individual lymph nodes from black, compared to white, patients harbored 4-fold greater quantities of metastatic tumor cells (p<0.001; FIG. 10) identified by GUCY2C RT-qPCR. Moreover, black patients harbored a greater burden of occult metastatic tumor across their lymph node network associated with the highest prognostic risk, compared to white patients (p=0.007; FIG. 10). Multivariate analyses revealed that black patients exhibited occult tumor burden associated with the greatest prognostic risk regardless of T stage or number of lymph nodes collected (FIG. 11).

Occult Tumor Burden is an Independent Prognostic Variable Associated with Racial Disparities in Outcomes.

Multivariable analyses employing polytomous logistic regression (Table 3) confirmed that race (p=0.02), T stage (p=0.02), and number of lymph nodes collected (p=0.003) are independently associated with occult tumor burden and stratification into risk categories (Table 3). Patients with T3/T4 tumors were more likely to be categorized as high risk versus low risk (adjusted odds ratio 6.00 [1.69-21.39]; p=0.006) compared to patients with T1/T2 tumors. Similarly, patients providing 13 or more lymph nodes were more likely to be categorized as high risk (adjusted odds ratio 8.10 [1.31-∞]; p=0.02) compared to patients with fewer lymph nodes collected. Importantly, black patients were more likely to be categorized as high risk on the basis of occult tumor burden compared to white patients (adjusted odds ratio 5.08 [1.69-21.39] p=0.006).

### Discussion

There is a well-established racial disparity in disease mortality in black, compared to white, patients with colorectal cancer. The data indicate that black, compared to white, patients exhibit higher levels of occult metastatic tumor cells in regional lymph nodes. These metastases are associated with a greater proportion of black, compared to white, patients harboring higher levels of occult tumor burden across their regional lymph node networks. In turn, this occult tumor burden is associated with racial disparities in stage-specific prognostic risk. Indeed, occult tumor burden was an independent marker of excess prognostic risk in black patients. These analyses further confirm the contribution of stage-specific differences in outcomes to racial disparities in overall mortality in colorectal cancer in the context of a prospective multicenter trial. They suggest that racial disparities in mortality, in part, reflect differences in clinically undetected tumor metastasis in black, compared to white, patients, revealed by occult tumor burden in regional lymph nodes. Importantly, this study suggests that quantifying occult tumor burden in regional lymph nodes can identify patients, regardless of race, that are at greatest risk for developing recurrent disease.

Stage-specific racial disparities in outcomes in pN0 black, compared to white, patients with colorectal cancer is associated with greater occult tumor burden in regional lymph nodes. These results demonstrate the impact of occult tumor burden on racial disparities in clinical prognosis.

### Example 4

There is an established relationship between the number of lymph nodes analyzed by histopathology and the accuracy of staging in colorectal cancer. While molecular approaches to identifying occult tumor cells are emerging, the relationship between the number of lymph nodes analyzed and the accuracy of staging has not yet been explored. Moreover, beyond the categorical (yes/no) identification of occult tumor cells in individual nodes, the number of nodes assessed may be relevant to the accuracy of quantifying occult tumor burden across the regional lymph node network. The present analysis identifies the relationship between the number of lymph nodes analyzed by GUCY2C quantitative (q)RT-PCR and the accuracy of risk stratification by estimating occult tumor burden in pN0 colorectal cancer patients.

Data from the study described in Example 2 was used in a subsequent analysis to explore the relationship between the number of lymph nodes analyzed and the accuracy of risk stratification based upon occult tumor burden in pN0 colorectal cancer patients. The data in Example 2 refers to analysis of lymph nodes from 291 patients. Of the 291 eligible patients, 23 were identified by their medical record as black, 259 as white and 9 were of another race or their race could not be identified. These analyses exploring the impact of the number of lymph nodes analyzed on the accuracy of risk stratification disclosed here exclude the data from the nine patients not identified as black or white and focus on the 282 patients identified as being black or white.

Thus, lymph nodes (range: 2-159) from 282 prospectively enrolled pN0 colorectal cancer patients were analyzed by GUCY2C quantitative (q)RT-PCR and followed for a median of 24 months (range: 2-63). Prognostic risk categorization defined using occult tumor burden was the primary outcome measure. Association of prognostic variables and risk were defined by multivariate polytomous and semi-parametric polytomous logistic regression. Occult tumor burden stratified this pN0 cohort into categories with low (60%; recurrence rate (RR)=2.3% [95% CI 0.1-4.5%]), intermediate (31%; RR=33.3% [23.7%-44.1%]), and high (9%; RR=68.0% [46.5%-85.1%], p<0.001) risk. Race, T stage and the number of lymph nodes collected for histopathology were independent markers of risk stratification. In that context, there was a direct relationship between the number of lymph nodes collected for histopathology and the number analyzed by GUCY2C qRT-PCR (p<0.001). Multivariable analysis revealed that the number of nodes analyzed by qRT-PCR was an independent prognostic marker of risk stratification (p<0.001). Indeed, occult tumor burden provided nearly complete resolution of risk categories in the heterogeneous pN0 population with >13 analytic lymph nodes. The prognostic accuracy of occult tumor burden assessed by GUCY2C qRT-PCR is dependent on the number of analytic lymph nodes with the greater number analyzed correlative to the accuracy.

### Methods

The basic study design, patients and tissues, RNA isolation, and RT-PCR are disclosed in Example 2. As noted above, the initial analysis of the lymph nodes available from the 299 criteria eligible pN0 patients resulted in eight patients being excluded from the study due to RNA of insufficient integrity by β-actin (two patients) and GUCY2C expression in tumors was below background levels (six patients). The analysis in Example 2 is thus based upon data from 291 patients. Data from nine additional patients was excluded in this subsequent analysis directed at impact of the number of lymph nodes analyzed to the accuracy of the risk stratification; the nine patients excluded were not identified as white or black. The analyses were performed on data from the 282 patients identified as white or black.

### Statistical Methods.

The primary clinical endpoint was molecular risk category (low, intermediate, high) based on time to recurrence and recursive partitioning analysis. Previous analyses of risk categories by polytomous logistic regression included an established standard cut-off for the number of harvested lymph nodes. Here, this model included the number of lymph nodes available for molecular analysis. Models were compared based on the Akaike Information Criteria (AIC=2k−2 ln(L)), where k is the number of parameters and L is the maximized value of the Likelihood of the estimated model), an established metric for the comparison of non-nested models (Bozdogan, H. Model selection and Akaike's information criterion (AIC): The general theory and its analytical extensions. Psychometrika, 52: 345-370, 1987.). Multivariable analyses were completed using semi-parametric polytomous logistic regression (Biesheuvel, C. J., Vergouwe, Y., Steyerberg, E. W., Grobbee, D. E., and Moons, K. G. Polytomous logistic regression analysis could be applied more often in diagnostic research. J Clin Epidemiol, 61: 125-34, 2008; Yee, T. W. The VGAM package for categorical data analysis. Journal of Statistical Software, 32: 1-34, 2010) to define the relationship between risk level and number of analytic lymph nodes. Inference for this modeling approach is not incorporated in the software and properties are as yet undetermined. Thus, 5,000 bootstrap samples are utilized to compute confidence intervals and empirical p values. Confidence intervals for raw survival rates were computed by the exact method of Clopper-Pearson (Newcombe, R. G. Two-sided confidence intervals for the single proportion: comparison of seven methods. Stat Med, 17: 857-72, 1998). All tests were two-sided, and p<0.05 was considered statistically significant. All analyses were performed with R v 2.11.2, SAS v9.2.

### Results

### Patient Characteristics.

The 282 pN0 patients had a mean age of 68 years (26-90 years) at diagnosis and 56% were male. Clinicopathologic features, including depth of tumor penetration (T1/2, T3, T4), and tumor anatomical location (right, left, rectal) were similar to national experience. Patients with colon cancer represented 86%, while those with rectal tumors comprised 14%. Black patients comprised 8.2% of the total population enrolled, nearly identical to the national average for disease-specific racial distribution. In this cohort, 77 (27%) patients provided <13 lymph nodes, 70 (25%) patients 14-21 lymph nodes, and 135 (48%) patients >22 lymph nodes for histopathology. There were no significant differences in clinicopathologic characteristics between patients providing different numbers of.

### Occult Tumor Burden and Risk Stratification.

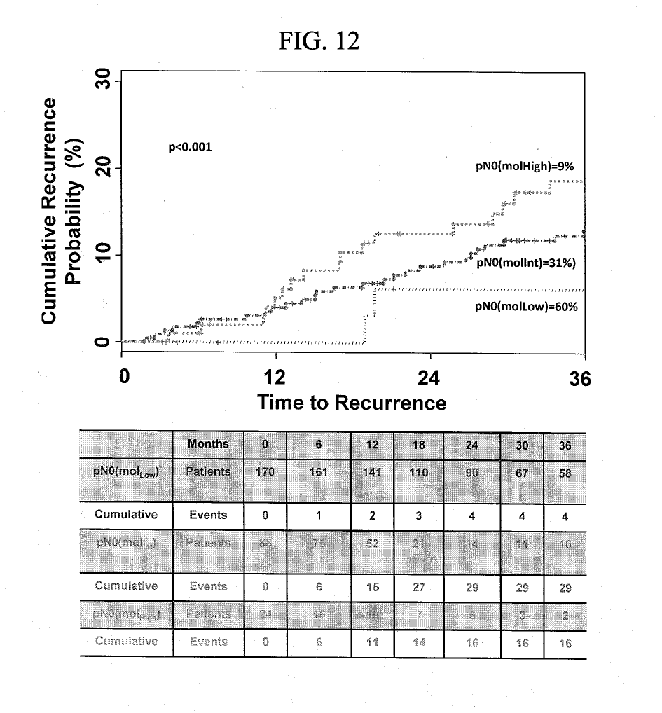

Clinical outcomes in pN0 colorectal cancer patients were analyzed by recursive partitioning using metrics of occult tumor burden estimated by GUCY2C qRT-PCR. Based on time to recurrence, GUCY2C qRT-PCR stratified pN0 patients into categories in which 170 (60%) patients exhibited low (MolLow), 88 (31%) exhibited intermediate (MolInt), and 24 (9%) exhibited high (MolHigh) (p<0.001) risk of disease recurrence (FIG. 12). All but 4 of the MolLow patients remained free of disease during follow-up (recurrence rate (RR)=2.3% [95% CI 0.1-4.5%]); 29 MolInt patients developed recurrent disease (RR=33.3% [23.7%-44.1%]); and 16 RR=68.0% [46.5%-85.1%]) MolHigh patients developed recurrent disease (p<0.001; FIG. 12). Univariate analysis revealed the expected relationship between advanced T stage, occult tumor burden and risk (p=0.008). Similarly, black patients harbored a greater burden of occult metastatic tumor across their lymph node network associated with the highest prognostic risk, compared to white patients (p=0.007).

### Occult Tumor Burden and Lymph Node Collections.

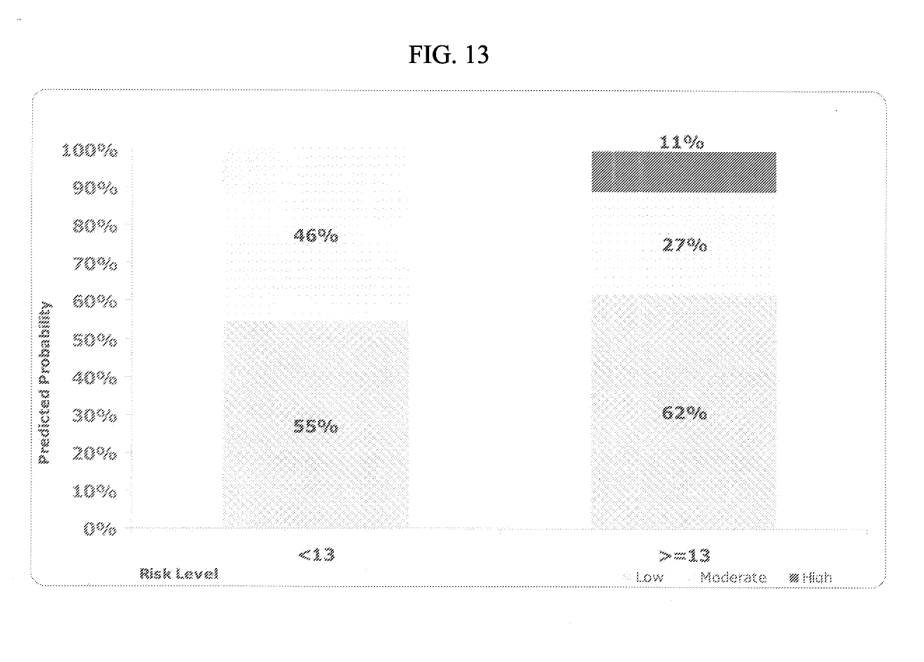

Surprisingly, the accuracy of molecular staging depended on the number of lymph nodes collected for histopathology. Patients providing fewer than 14 lymph nodes exhibited occult tumor burdens that stratified patients in low and intermediate risk categories, with only 6.5% of patients in the highest risk category. Conversely, analysis of >14 lymph nodes minimized the number of patients with intermediate risk while maximizing patients with the lowest and highest risk (p<0.001; FIG. 13). Indeed, collection of >14 lymph nodes for histopathology was associated with a 3-fold enhancement in identifying patients with the greatest prognostic risk. This association of staging accuracy by qRT-PCR with increased lymph node collections recapitulates established improvements in histopathologic staging by increased nodal harvests.

The 282 eligible pN0 patients provided 6,699 lymph nodes (range 2-159, median 21 lymph nodes/patient) for histopathologic examination, of which 2,570 (range 1-33, median 8 lymph nodes/patient) were eligible for analysis by qRT-PCR. The greater number of lymph nodes available for histopathology, compared to molecular analysis, from pN0 patients includes those collected after formalin fixation or nodes<5 mm in diameter, smaller than the limit of bisection. Association between accuracy of staging by occult tumor burden and number of nodes collected for histopathology suggested a relationship between total nodal harvest and nodes analyzed by qRT-PCR. Indeed, there was a direct association between the number of lymph nodes collected for histopathology and those provided for qRT-PCR (R=0.49, p<0.001; FIG. 13). Moreover, the accuracy of molecular staging depended on the number of lymph nodes analyzed by qRT-PCR. Thus, patients providing <13 analytic nodes exhibited occult tumor burden that stratified patients in low and intermediate risk categories, with few patients (7%) in the highest risk category (FIG. 13). Conversely, >14 lymph nodes undergoing molecular analysis improved prognostic resolution, reducing the number of patients in the intermediate risk category while maximizing the identification of patients with the lowest and highest risk (p<0.001).

### Occult Tumor Burden is an Independent Prognostic Variable Defined by Number of Analytic Lymph Nodes.

Multivariable analyses employing polytomous logistic regression confirmed that race, T stage, and number of analytic lymph nodes assessed by qRT-PCR are independently associated with quantification of occult tumor burden and stratification into risk categories. Black patients were more likely to be categorized as high risk on the basis of occult tumor burden compared to white patients (adjusted odds ratio 4.05 [1.01-16.67] p=0.03). Similarly, patients with T3 tumors were more likely to be categorized as high risk (adjusted odds ratio 5.51 [2.15-31.10]; p<0.001) compared to patients with T1/T2 tumors. Importantly, the number of analytic lymph nodes was essential to accurately stratify risk by occult tumor burden (p<0.001). Indeed, using >13 lymph nodes to quantify occult tumor burden categorized ˜70% of pN0 patients with low risk and ˜30% of patients with intermediate and high risk. Moreover, using >25 lymph nodes to quantify occult tumor burden almost completely resolved these latter categories, stratifying almost all patients who were not low risk as high risk and nearly eliminating the intermediate risk category.

### Discussion

While quantification of occult tumor burden offers a previously unavailable opportunity to identify patients at risk in the prognostically heterogeneous pN0 population, this staging paradigm classifies ˜30% of patients as having intermediate risk. Intermediate risk could reflect variations in tumor biology which influence recurrence beyond the quantity of occult tumor burden in lymph nodes. Alternatively, systematic misclassification of high or low risk patients into the intermediate risk category could result from inaccurate quantification of occult tumor burden in inadequate collections of analytic lymph nodes. There is a well-established relationship between the accuracy of conventional staging and the number of lymph nodes analyzed by histopathology. Increased lymph node collections improve the likelihood of identifying macroscopic tumor deposits by histopathology, which depends on limited tissue sampling techniques. In the context of molecular paradigms employing GUCY2C qRT-PCR, increased analytic lymph node collections improve the accuracy of occult tumor burden quantification across the regional lymph node network. This is underscored by the observation that quantification of occult tumor burden employing very small numbers of analytic lymph nodes only identifies patients with low or intermediate risk, but fails to identify patients with high risk (FIG. 3). In striking contrast, analyzing >25 lymph nodes nearly eliminates the intermediate risk category, classifying almost all patients in low or high risk groups.

Current practice guidelines recommend the collection of >12 lymph nodes to optimize staging of colorectal cancer patients by conventional approaches. In contrast, the present results suggest that analyzing >25 lymph nodes for occult tumor burden provides nearly complete resolution of risk classification in the pN0 population. This approach identified ˜70% of patients with near-zero risk, while ˜30% of patients were classified with high risk. This recapitulates the true risk of this population, in which ˜70% of pN0 patients remain disease-free while up to ˜30% of patients ultimately develop recurrent disease. It is noteworthy that this level of accuracy, with near-complete resolution of risk stratification, has not been achieved previously for pN0 colorectal cancer patients.

While analysis of >25 lymph nodes provides the most accurate classification of risk, the data suggest that patient management can be optimized using >13 lymph nodes. Analysis of 13 lymph nodes provides optimum resolution of patients with low risk and those who do not have low risk. Adding more lymph nodes to the analysis only improves the accuracy of classifying patients with high risk who were otherwise misclassified with intermediate risk, without further improving the classification of low risk patients. The utility of >13 lymph nodes to optimally classify patients with low risk and those who do not have low risk (consequently, high risk) by occult tumor burden analysis suggests that this emerging molecular paradigm is compatible with current recommendations guiding lymph node collection.

The present observations demonstrate that the accuracy of staging pN0 colorectal cancer patients by occult tumor burden analysis employing GUCY2C qRT-PCR is dependent on the number of analytic lymph nodes. They suggest that with >13 lymph nodes for the analysis, occult tumor burden can provide near complete resolution of prognostic risk stratification in the otherwise heterogeneous pN0 cohort. These studies suggest a near absolute relationship between the amount of tumor deposits in regional lymph nodes and the risk of metastatic disease. They underscore the importance of lymphatic spread of colorectal cancer as an essential process in tumor dissemination and metastatic disease. Most importantly, the ability of occult tumor burden analysis to near-completely resolve prognostic risk offers an unprecedented opportunity to identify patients who could most benefit from adjuvant treatment in the otherwise therapeutically ambiguous pN0 population.

TABLE 1
Characteristics of pN0 Patients with Colorectal Cancer

Variable

| Lymph Nodes | N | % |
|---|---|---|
| Totals | 291 | 100 |
| Age, years | | |
| <50 | 25 | 8.6 |
| 50-75 | 186 | 63.9 |
| >75 | 80 | 27.5 |
| Sex | | |
| Male | 160 | 55.0 |
| Female | 131 | 45.0 |
| T Stage | | |
| T1/T2 | 120 | 41.2 |
| T3 | 151 | 51.9 |
| T4 | 20 | 6.9 |
| Grade | | |
| Well | 20 | 6.9 |
| Moderate | 226 | 77.7 |
| Poor/unknown | 45 | 15.4 |
| Chemotherapy | | |
| Yes | 65 | 22.3 |
| No | 226 | 77.7 |
| Tumor Site | | |
| Left Colon | 19 | 6.5 |
| Right Colon | 119 | 40.9 |
| Sigmoid Colon | 112 | 38.5 |
| Rectum | 41 | 14.1 |
| Nodes Harvested | | |
| <12 | 48 | 16.5 |
| ≧12 | 242 | 83.5 |
| Lymphovascular Invasion | | |
| No | 233 | 80.1 |
| Yes | 58 | 19.9 |

TABLE 2

| | | Black | White | |
|---|---|---|---|---|
| | Overall | (n = 23) | (n = 259) | |
| Characteristic | n | %‡ | %‡ | p† |
| Age at Diagnosis | | | | 0.55 |
| <65 | 107 | 43.5 | 37.1 | |
| ≧65 | 175 | 56.5 | 62.9 | |
| Sex | | | | 0.22 |
| Male | 157 | 43.5 | 56.0 | |
| Female | 125 | 56.5 | 44.0 | |
| Location | | | | 0.27 |
| Left | 128 | 35.1 | 46.3 | |
| Right | 115 | 56.5 | 39.4 | |
| Rectal | 39 | 8.7 | 14.3 | |
| Differentiation | | | | 0.49 |
| Poor/unknown | 45 | 13.0 | 16.2 | |
| Moderate | 217 | 74.0 | 77.2 | |
| Well | 20 | 13.0 | 6.6 | |
| T Stage | | | | 0.26 |
| T1/T2 | 117 | 30.5 | 42.5 | |
| T3/T4 | 165 | 69.5 | 57.5 | |
| Lymphovascular Invasion | | | | 0.35 |
| No | 224 | 87.0 | 79.5 | |
| Yes | 58 | 13.0 | 20.5 | |
| Treatment | | | | 0.69 |
| Surgery alone | 218 | 73.9 | 77.7 | |
| Surgery + chemotherapy | 64 | 26.1 | 22.3 | |
| Nodes Harvested | | | | 0.69 |
| <13 | 59 | 17.4 | 20.9 | |
| ≧13 | 223 | 82.6 | 79.1 | |

†P value from chi-square test of association.
‡% of total for race.

TABLE 3

| | | Adjusted | | |
|---|---|---|---|---|
| | Overall | Odds Ratio | 95% | |
| Characteristic | n | (AOR) | CI | P † |
| Race | | | | 0.02 ‡ |
| White | 259 | Referent | — | |
| Black | 23 | 1.03 | (0.36, 2.94) | 0.95 † |
| (Moderate vs Low) | | | | |
| Black | | 5.08 | (1.55, 16.65) | 0.007 † |
| (High vs Low) | | | | |
| T Stage | | | | 0.02 ‡ |
| T1/T2 | 117 | Referent | — | |
| T3/T4 | 165 | 1.25 | (0.74, 2.12) | 0.41 † |
| (Moderate vs Low) | | | | |
| T3/T4 | | 6.00 | (1.69, 21.39) | 0.006 † |
| (High vs Low) | | | | |
| Nodes Harvested * | | | | 0.003 ‡ |
| <13 | 59 | Referent | — | |
| ≧13 | 223 | 0.43 | (0.28, 1.01) | 0.06 † |
| (Moderate vs Low) | | | | |
| ≧13 | | 8.10 | (1.31, ∞) | 0.02 † |
| (High vs Low) | | | | |

† P value from multivariable logistic regression model, 1df Wald (exact) chi-square test.
‡ Type 3 overall (exact) test of association from multivariable logistic regression model.
* Indicates exact tests reported from multivariable model.

SEQUENCE LISTING

SEQ ID NO: 1 - ATTCTAGTGGATCTTTTCAATGACCA
SEQ ID NO: 2 - CGTCAGAACAAG-GACATTTTTCAT
SEQ ID NO: 3 - (FAM-TACTTGGAGGACAATGTCACAG-
CCCCTG-TAMRA)
SEQ ID NO: 4 - CCACACTGTGCCCATCTACG
SEQ ID NO: 5 - AGGATCTTCATGAG-GTAGTCAGTCAG
SEQ ID NO: 6 - (FAM-ATGCCC-X(TAMRA)-
CCCCCATGCCATCCTGCGTp)

1. A database for predicting clinical outcomes based upon quantitative tumor burden in lymph node samples from an individual, said database comprising data sets for a plurality of individuals which include clinical outcome data and data regarding number of lymph nodes evaluated, maximum number of biomarker detected in any single node, median normalized expression levels detected across all evaluated lymph nodes and the maximum normalized expression levels detected in any evaluated lymph nodes; said database also providing stratified risk categories based upon recursive partitioning of data.

2. The database of claim 1 wherein each data set from each individual has data from at least 14 lymph nodes evaluated for quantitative tumor burden.

3. The database of claim 1 wherein the quantitative tumor burden is assessed by RT-PCR.

4. The database of claim 1 wherein the quantitative tumor burden is determined by quantifying the biomarker GCC or a nucleic acid sequence molecule encoding GCC.

5. A system for predicting clinical outcomes based upon quantitative tumor burden in lymph node samples from an individual comprising,
a database of claim 1;
an input interface to input a test patient data set including data regarding number of lymph nodes evaluated, maximum number of biomarker detected in any single node, median normalized expression levels detected across all evaluated lymph nodes and the maximum normalized expression levels detected in any evaluated lymph nodes;
a data processor for processing inputted patient data with data in the database, wherein said processing assigns said test patient data to a stratified risk category; and
an output interface which displays test patients identity and assigned stratified risk category.

6. The system of claim 5 wherein the output interface comprises a printer which prints a report containing test patient identity information and assigned stratified risk category.

7. The system of claim 5 wherein the output interface comprises an electronic data generator which generates an electronic report containing test patient identity information and assigned stratified risk category.

8. The system of claim 5 wherein each data set in the database is a data set from an individual that has data from at least 14 lymph nodes evaluated for quantitative tumor burden.

9. The system of claim 5 wherein the quantitative tumor burden is assessed by RT-PCR.

10. The system of claim 5 wherein the quantitative tumor burden is determined by quantifying the biomarker GCC or a nucleic acid sequence molecule encoding GCC.

11. A method of preparing a database of claim 1 comprising
compiling data sets for a plurality of individuals which include clinical outcome data and data regarding number of lymph nodes evaluated, and an output interface to maximum number of biomarker detected in any single node, median normalized expression levels detected across all evaluated lymph nodes and the maximum normalized expression levels detected in any evaluated lymph node; and
processing said data sets using recursive partitioning to produce stratified risk categories.

12. The method of claim 11 wherein said data sets are processed using recursive partitioning to produce stratified risk categories by
first partitioning data sets based upon maximum copies on any node wherein data sets are divided into a high group and a low group;
partitioning data sets in said high group and said low group into four groups based upon median normalized expression levels detected across all evaluated lymph nodes to divide said high group into a high low group and a high-high group and to divide said low group into a low-low group and a low-high group;
partitioning data sets in said high-high group and said low-high group into four groups based upon maximum normalized expression levels detected in any evaluated lymph nodes to divide said high-high group into a high-high-high group and a high-high-low group and to divide said low-high group into a low-high-low group and a low-high-high group; thereby partitioning said data sets into six groups total, 1) high-low, 2) high-high-low, 3) high-high-high, 4) low-low, 5) low-high-high, and 6) low-high-low;
comparing outcomes associated with each data set in each group to determine risk categories, wherein
1) high-low, 2) high-high-low, and 4) low-low are low risk;
5) low-high-high is high risk; and
3) high-high-high and 6) low-high-low are independently assigned low, medium or high based upon outcome.

13. The method of claim 11 wherein 1) high-low, 2) high-high-low, 4) low-low and 6) low-high-low are low risk; and 3) high-high-high and 5) low-high-high are high risk.

14. The system of claim 11 wherein each data set in the database is a data set from an individual that has data from at least 14 lymph nodes evaluated for quantitative tumor burden.

15. The system of claim 11 wherein the quantitative tumor burden is assessed by RT-PCR.

16. The database of claim 11 wherein the quantitative tumor burden is determined by quantifying the biomarker GCC or a nucleic acid sequence molecule encoding GCC.

17. A method for predicting clinical outcome for a test patient or a group of test patients based upon quantitative tumor burden in lymph node samples from an individual or group of individuals comprising:
measuring quantitative tumor burden in a plurality of lymph node samples from an individual or group of individuals;
inputting said data into a system of claim 5;
processing inputted data in database of said system, wherein said processing assigns said data test patient to a stratified risk category; and produces an output that displays test patient's identity and assigned stratified risk category.

18. The method of claim 17 wherein each data set in the database is a data set from an individual that has data from at least 14 lymph nodes evaluated for quantitative tumor burden.

19. The method of claim 17 wherein the quantitative tumor burden is assessed by RT-PCR.

20. The method of claim 17 wherein the quantitative tumor burden is determined by quantifying the biomarker GCC or a nucleic acid sequence molecule encoding GCC.

21. A method for predicting clinical outcome for a test patient or a group of test patients based upon quantitative tumor cell burden in lymph node samples from test patient or group of test patients comprising:
using recursive partitioning to produce stratified risk categories associated with the quantitative tumor cell burden in a plurality of lymph node samples from the test patient or group of test patients.

22. The method of claim 21, wherein, prior to the step of using recursive partitioning to produce stratified risk categories associated with the quantitative tumor cell burden in a plurality of lymph node samples from the test patient or group of test patients, the method comprises the steps of:
generating a data set based upon a plurality of lymph node samples from a test patient or group of test patients; and
inputting the data set into the database of claim 1.

23. The method of claim 22, wherein the step of generating a data set comprises measuring the quantity of GCC in the lymph node samples.

24. The method of claim 22 wherein the tumor burden is generated by quantifying the biomarker GCC or a nucleic acid sequence molecule encoding GCC by quantitative PCR.

25. The method of claim 22, wherein the step of generating a data set comprises measuring the quantity of GCC in the lymph node samples of the patient or the group of patients and measuring the quantity of at least one other biomarker associated with a tumor cell.


SEQUENCE LISTING (6 sequences)

SEQ ID NO: 1 - DNA, 26 bp, Artificial Sequence
  PCR PRIMERS
attctagtggatcttttcaatgacca

SEQ ID NO: 2 - DNA, 24 bp, Artificial Sequence
  PCR PRIMERS
cgtcagaacaaggacatttttcat

SEQ ID NO: 3 - DNA, 35 bp, Artificial Sequence
  RT-PCR PRIMERS
amtacttggaggacaatgtcacagcccctgtamra

SEQ ID NO: 4 - DNA, 20 bp, Artificial Sequence
  RT-PCR PRIMERS
ccacactgtgcccatctacg

SEQ ID NO: 5 - DNA, 26 bp, Artificial Sequence
  RT-PCR PRIMERS
aggatcttcatgaggtagtcagtcag

SEQ ID NO: 6 - DNA, 32 bp, Artificial Sequence
  RT-PCR PRIMERS
amatgccctamracccccatgccatcctgcgt